(19) United States
(12) Patent Application Publication
(10) Pub. No.: US 2023/0190314 A1
(43) Pub. Date: Jun. 22, 2023

(54) ASPIRATION SYSTEM INCLUDING THROMBUS-DISRUPTING INNER MEMBER

(71) Applicant: Covidien LP, Mansfield, MA (US)

(72) Inventors: Wanda F. Dent, Chanhassen, MN (US); Anthony J. O'Connor, Chaska, MN (US); Katherine L. Stevenson, Minneapolis, MN (US); Niall F. Duffy, Galway (IE); Edward C. Stankard, Oranmore (IE); Graham Robert Cole, Roscrea (IE); Dion Gallagher, Urlanbeg (IE)

(21) Appl. No.: 18/066,589

(22) Filed: Dec. 15, 2022

Related U.S. Application Data
(60) Provisional application No. 63/265,683, filed on Dec. 17, 2021.

(51) Int. Cl. A61B 17/22

(52) U.S. Cl. CPC A61B 17/22; A61B 2017/22079; A61B 2217/005

(57) ABSTRACT

An example aspiration system includes an inner member configured to be received within an aspiration catheter lumen and disrupt a thrombus positioned within vasculature of a patient. The inner member includes a distal member at a distal portion of an elongated support member, where the distal member is configured to fit within the aspiration catheter lumen. The distal member defines a larger cross-sectional dimension than the elongated support member, the cross-section being taken in a direction orthogonal to a longitudinal axis of the support member, and is configured to be deployed distally outward from a distal opening of the aspiration catheter lumen to contact and disrupt a thrombus to facilitate the efficiency of a medical aspiration procedure. The distal member may include one or more tapered portions and a surface disruption configured to aid with the thrombus disruption.

This application claims the benefit of U.S. Provisional Application No. 63/265,683, filed Dec. 17, 2021, and entitled, “ASPIRATION SYSTEM INCLUDING THROMBUS-DISRUPTING INNER MEMBER,” the entire content of which is incorporated herein by reference.

### TECHNICAL FIELD

This disclosure relates to medical devices.

### BACKGROUND

A medical catheter defining at least one lumen has been proposed for use with various medical procedures. For example, in some cases, a medical catheter may be used to access and treat defects in blood vessels, such as, but not limited to, lesions or occlusions in blood vessels.

### SUMMARY

This disclosure describes example aspiration systems that include an inner member configured to be received within an aspiration catheter lumen and disrupt (e.g., penetrate, segment, break-up, compress, or the like) a thrombus positioned within vasculature of a patient. The inner member includes a distal member at a distal portion of an elongated support member, where the distal member is configured to fit within the aspiration catheter lumen. The distal member defines a larger cross-sectional dimension than the elongated support member, the cross-section being taken in a direction orthogonal to a longitudinal axis of the support member, and is configured to be deployed distally outward from a distal opening of the aspiration catheter lumen to contact and disrupt a thrombus, which can help facilitate the efficiency of a medical aspiration procedure. The distal member may include one or more tapered portions and a surface disruption configured to aid with the thrombus disruption. For example, the surface disruption can include a groove or a protrusion.

In some examples, the distal member may be alternatingly distally deployed and proximally withdrawn to repeatedly contact and disrupt a thrombus during an aspiration procedure. A suction force may be applied to the catheter lumen to aspirate the thrombus (or portions of the thrombus) into the distal opening of the catheter for removal from the vasculature.

In one example, this disclosure describes a medical system including: an aspiration catheter defining a catheter lumen; and an inner member configured to be received in the catheter lumen and extend distally outward from a distal opening of the catheter, wherein the inner member comprises: an elongated support member configured to move axially within the catheter lumen, the elongated support member defining a longitudinal axis; and a distal member at a distal portion of the elongated support member, wherein the distal member defines a larger cross-sectional dimension than the elongated support member in a direction orthogonal to the longitudinal axis, wherein a proximal-facing surface of the distal member defines a proximal taper, and wherein the distal member defines a surface disruption configured to aid disruption of a thrombus.

In another example, this disclosure describes a method including: deploying a distal member of an inner member from a distal opening of an aspiration catheter and contacting a thrombus with the distal member, the aspiration catheter defining a catheter lumen, wherein the inner member comprises: an elongated support member configured to move axially within the catheter lumen, the elongated support member defining a longitudinal axis; and the distal member at a distal portion of the elongated support member, wherein the distal member defines a larger cross-sectional dimension than the elongated support member in a direction orthogonal to the longitudinal axis, wherein a proximal-facing surface of the distal member defines a proximal taper, and wherein the distal member defines a surface disruption configured to aid disruption of the thrombus; and moving, via the elongated support member, the distal member at least one of proximally or distally through the thrombus.

In another example, this disclosure describes an article including: a member configured to be received in a lumen of an aspiration catheter and extend distally outward from a distal opening of the aspiration catheter includes an elongated support member configured to move axially within the catheter lumen; and a distal member at a distal portion of the elongated support member, wherein the distal member defines a larger cross-sectional dimension than the elongated support member in a direction orthogonal to the longitudinal axis, wherein a proximal-facing surface of the distal member defines a proximal taper, wherein the distal member defines a surface disruption configured to aid disruption of a thrombus.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1A is a schematic diagram illustrating an example medical aspiration system.

FIG. 1B is a schematic diagram illustrating another example medical aspiration system.

FIG. 2 is a conceptual side view of an example aspiration catheter and an example inner member of the aspiration systems of FIGS. 1A and 1B.

FIG. 3A is a conceptual cross-sectional view of an example inner member, where the cross-section is taken through a center of the inner member and along a longitudinal axis of the inner member.

FIG. 3B is a conceptual cross-sectional view of an example inner member including a distal member extended out of a distal opening of a catheter, where the cross-section is taken through a center of the inner member and along a longitudinal axis of the inner member.

FIG. 4A is a conceptual cross-sectional view of an example biased inner member, where the cross-section is taken through a center of the biased inner member and along longitudinal axes of the biased inner member.

FIG. 4B is a conceptual cross-sectional view of the example biased inner member of FIG. 4A with the distal member advanced through the distal opening of a catheter.

FIG. 5 is a conceptual cross-sectional view of an example inner member.

FIG. 6 is a conceptual cross-sectional view of a part of an example distal member of the inner member of FIG. 5.

FIGS. 7A-7G are conceptual side views of example distal members.

FIGS. 7H-7L are conceptual cross-sectional views of example distal members.

FIG. 8 is a conceptual side view of a distal portion of another example inner member.

FIG. 9 is a flow diagram of an example method of using the example catheter.

FIG. 10 is a flow diagram of another example method of using the example catheter.

### DETAILED DESCRIPTION

The present disclosure describes devices, systems, and methods related to aspirating a thrombus within a vasculature of a patient. The disclosure describes example aspiration systems that include an aspiration catheter and a mechanical “clot buster,” such as an inner member configured to be received within a lumen of an aspiration catheter and including a distal member (e.g., a bulb or other enlarged structure) fixed on a distal portion of an elongated support member (e.g., a polymer and/or metal wire, such as a solid-core Nitinol wire). The inner member is configured to be longitudinally deployable via the lumen of the aspiration catheter and the distal member is configured to engage with a thrombus (e.g., a blood clot or other material such as a plaques or foreign bodies) positioned within vasculature of a patient, in order to facilitate aspiration and removal of the thrombus from the vasculature. The inner member is configured to move axially (e.g., proximally and distally) relative to the aspiration catheter (also referred to herein more generally as a catheter) in order to disrupt and/or penetrate the thrombus. Disruption of the thrombus by the inner member may compress the thrombus into a smaller volume and/or break the thrombus up into smaller segments, which may facilitate aspiration of the thrombus through the lumen of the aspiration catheter (referred to herein as a catheter lumen or an aspiration catheter lumen). For example, a clinician may alternatingly distally advance and proximally withdraw the distal member of the inner member relative to the aspiration catheter to repeatedly contact and segment the thrombus while the suction force is applied to the catheter lumen.

A distal member at a distal portion of the inner member includes a surface disruption configured to aid disruption of a thrombus. The surface disruption, which may also be referred to as a thrombus and/or clot disruption feature in some examples, can include, for example, a surface protrusion (e.g., a ridge) and/or a surface recess (e.g., a groove). The surface disruption can, for example, increase a surface area of the distal member, which increases the amount of engagement between the distal member and the thrombus. The surface disruption may provide one or more edges configured to disrupt a thrombus, e.g., during distal and/or proximal movement of the distal member relative to the aspiration catheter. In some examples, the surface disruption may provide a tactile response (e.g., tactile feedback) to a clinician, e.g., indicating engagement between the distal member and a thrombus, which can enable medical imaging (e.g., fluoroscopy) to be turned off for part of the medical aspiration procedure.

In examples described herein, the distal member of the inner member defines a proximal taper, e.g., defining one or more proximal tapered portions. Thus, on a proximal portion of the inner member, the inner member defines one or more surfaces that taper towards an outer surface of the elongated support member. The proximal taper may be configured to provide a tactile response along the inner member during use of the aspiration system. For example, the tactile response can be indicative of a position of the distal member relative to a distal opening of the aspiration catheter or relative to a thrombus. Thus, in some examples, the proximal tapered portion of the distal member improves the ability of the clinician to determine the position of the distal member relative to the distal opening of the aspiration catheter and/or the thrombus via tactile feel. In some examples, the proximal taper is configured to provide a tactile response while reducing negative impacts to the aspiration, such as reducing an amount of a loss of suction force and/or reducing turbulence of the flow into the catheter lumen and thereby reducing portions of the thrombus that may otherwise turbulently flow past the lumen rather than into it.

In some examples, the proximal taper providing a tactile response indicative of a position of the distal member relative to the distal opening of the catheter or relative to the thrombus may enable a clinician to manipulate the inner member, e.g., alternatingly advance and withdraw the distal member to repeatedly contact and segment the thrombus without medical imaging, which reduces the amount of radiation exposure to the patient. In addition, the proximal taper may facilitate disruption of a thrombus by providing a surface that dilates a pathway through a thrombus as the inner member is moved proximally towards the aspiration catheter.

In some examples, the distal member has a convex distal surface (e.g., a bulbous end). The convex distal surface may be an atraumatic distal end configured to reduce and/or minimize adverse impacts to a vessel wall when the distal member is positioned within vasculature of a patient. The inner member terminates at the convex distal surface in some examples, e.g., there is no further structure distal to the convex distal surface. The distal member can completely cover the distal end of the elongated support member, such that there is no wire or “tip” that extends beyond the distal end of the distal member. That is, the inner member distally terminates at the distal surface of the distal member.

The distal member is attached to a distal portion (e.g., a distal end) of the elongated support member. The inner member can include one or more structures (e.g. anchor) to help facilitate secure attachment between the distal member and the elongated support member. For example, in some examples in which the distal member is made of a polymer, the inner member includes a polymer layer between the elongated support member and the distal member to facilitate attachment of the distal member to the elongated support member and to reduce and/or prevent kinking and/or deformation of the elongated support member. The polymer of the distal member may bond and/or adhere better to the polymeric layer than directly to the elongated support member. The polymer layer can be, for example a polymeric coating applied to an outer surface of the elongated support member and/or a polymeric jacket (e.g., a tubular jacket) positioned on the outer surface of the elongated support member.

In addition to or instead of the polymer layer, the elongated support member may further include one or more retention members, e.g., mechanical attachment features such as, a bulb, a sphere, a ball, a coil, or the like or combinations thereof, that is attached onto (e.g., via soldering, laser cutting, swaging, or the link), or integrally formed with, the distal end of the elongated support member. The distal member may be formed over or otherwise positioned over the retention member and a length of the elongated support member and, if present, the polymeric layer, e.g., via molding or other suitable technique. The retention member is configured to secure and/or increase a retention force attaching the distal member to the elongated support member. For example, the retention member may be configured to increase a surface area of a distal portion of the elongated support member, e.g., thereby increasing the surface area for attaching and/or bonding with the distal member. The retention member may also provide a mechanical interlock when the bulb is molded over the retention member. The retention member can be formed from the same or a different material as the elongated support member or may be soldered or welded onto the elongated support member. In some examples, the retention member is a metal, such as Nitinol.

In some examples, the distal member or distal portion of the elongated support member includes a radiopaque material, e.g., a radiopaque coating over a portion of the elongated support member (e.g., such as the retention member), radiopaque particles dispersed within the polymer of the distal member and/or polymeric cover/coating, or the like.

In some examples, the elongated support member tapers in a distal direction, from a larger cross-sectional area (e.g., a larger diameter Nitinol wire) at a proximal end (e.g., the end outside the body and manipulable by the physician/user) to a smaller cross-sectional area at the distal end (e.g., to be more flexible to be advanced to the thrombus within the aspiration catheter while maintaining kink-resistance). The taper can be continuous or a stepwise taper. In some examples, the elongated support member may have multiple tapered portions. For example, the elongated support member may have a first tapered length to an intermediate diameter for an intermediate length, e.g., to increase the flexibility of the elongated support member traversing through the catheter, and a second tapered length to the distal diameter for a distal length, e.g., to increase flexibility and sizing at the distal end to allow an acceptable aspiration/suction force within the aspiration catheter that also contains the elongated support member.

In some examples, during an aspiration procedure, a thrombus may be aspirated to the distal opening of the aspiration catheter and may clog the distal opening or a part of the catheter lumen more proximal to the distal opening. The inner member is configured to help facilitate clearing of the distal opening and/or the catheter lumen during a medical procedure, e.g., without requiring the catheter to be removed from the patient. Enabling declogging of the catheter lumen without removal of the catheter from the patient may help limit the time required to complete a medical aspiration procedure. For example, to help clear the thrombus and enable further aspiration through the catheter lumen, a clinician may advance the elongated support member and distal member out of the distal end of the catheter to push away the thrombus, and/or to push through the thrombus. The clinician/user may then retract the elongated support member and distal member to disrupt the thrombus (e.g., compress and/or macerate the thrombus), with the aid of the surface disruptions of the distal member. The convex curved surface profile of the distal member may improve pushing of the thrombus and may reduce adverse impacts to a vessel wall, and the surface disruptions may help macerate or cause the distal member to lightly “grip” the thrombus as the distal member is moved back and forth, e.g., moving/agitating at least a portion of the thrombus.

In some examples, the clinician may advance the elongated support member and distal member out of the distal end of the catheter before the catheter engages the thrombus. The clinician may then advance the catheter, with the distal member advanced out of the catheter, to engage the thrombus. The clinician may then retract the elongated support member and distal member to disrupt the thrombus (e.g., compress and/or macerate the thrombus), e.g., without having to push the thrombus away from the distal end of the catheter before disrupting the thrombus.

In some examples, the clinician may use an actuator device, e.g., a switch, a slider, a knob, a torque device, or the like, to advance, retract, or otherwise move or manipulate the elongated support member. For example, the clinician may mount and/or attach a torque device and/or knob to a proximal portion of the elongated support member. For example, the elongated support member may be difficult to grab, e.g., elongated support member may be thin and may be slippery. The torque device may be configured to grip the elongated support member, and the clinician may manipulate the elongated support member by manipulating the torque device gripping the elongated support member.

In some examples, the clinician may use the elongated support member and/or distal member to determine a patency of the lumen of the catheter, e.g., whether the lumen is open, unobstructed, or not closed (patent) or whether the lumen is closed, obstructed, blocked and/or kinked (not patent). For example, the clinician may advance the distal member via the elongated support member through the catheter lumen and, if the lumen is not patent, the clinician may receive a tactile response from the elongated support member that the lumen is not patent, e.g., the distal member will no longer advance or may require an increased force to advance, and/or the elongated support member may bend or buckle due to the distal member no longer advancing through the lumen, or the like. In some examples, the clinician may make the lumen patent using the elongated support member and/or distal member, e.g., by straightening a kinked portion of the lumen by advancing the distal member through the lumen and the kinked portion to cause the kink to straighten.

In some examples, the distal member includes one or more openings configured to deliver a fluid from the distal member via the inner member. For example, a clinician may direct a flow of a fluid, such as saline, from a plurality of openings defined by a proximal-facing surface of the distal member, which may help direct portions of the thrombus into the distal opening of the catheter. In some examples, the proximally directed fluid flow delivered via the inner member is configured to dilute and/or displace a volume of incidental patient fluid, such as blood, that might otherwise be withdrawn from the patient through the catheter lumen during the aspiration procedure. When the distal member is proximally retracted back into the catheter lumen, the clinician may direct the flow of the fluid from the plurality of openings and proximally through the catheter lumen to flush the lumen of the catheter. Enabling flushing of the catheter without removal of the catheter from the patient may help limit the time required to complete a medical aspiration procedure. Further example details of proximally directing fluid flow from a distal member of an inner member configured to be received within a lumen of an aspiration catheter are found in U.S. Provisional Patent Application 63/253,679, filed Oct. 8, 2021, and entitled “Aspiration System Including Fluid-Infusing Inner Member.”

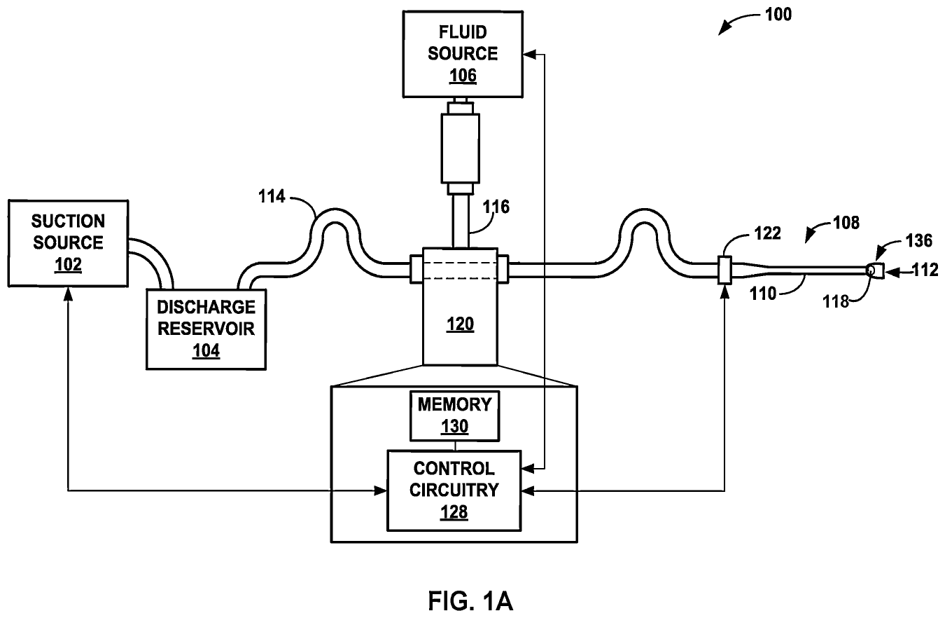

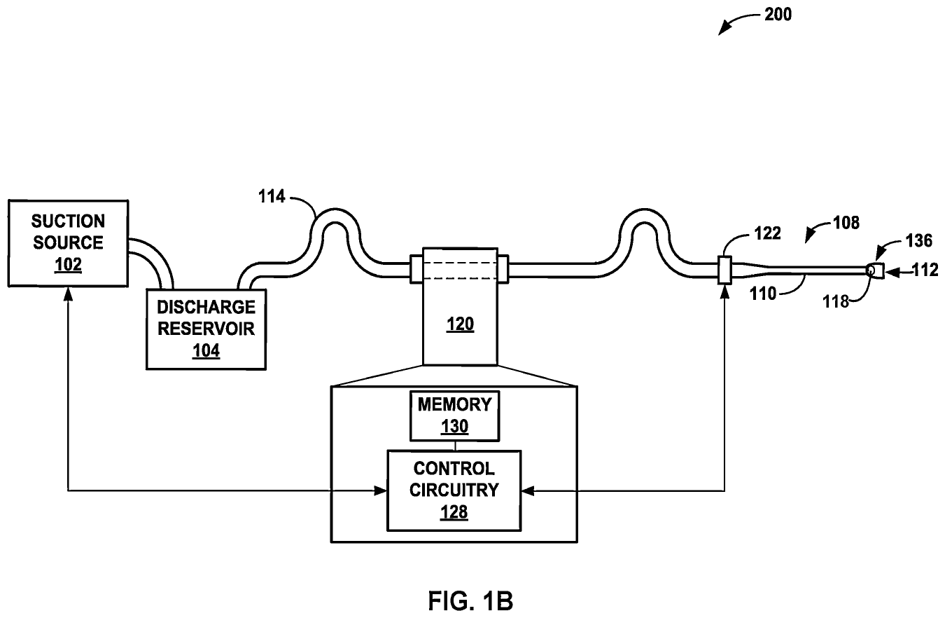

FIG. 1A is a schematic diagram illustrating an example medical system 100 including a suction source 102, a discharge reservoir 104, a fluid source 106, an aspiration catheter 108, and an inner member 118 configured to be received in a lumen of catheter 108. FIG. 1B is a schematic diagram illustrating another example medical system 200 that is substantially similar to medical system 100 of FIG. 1A, but without fluid source 106. Medical system 200 includes suction source 102, discharge reservoir 104, aspiration catheter 108, and inner member 118 configured to be received in the lumen of catheter 108. FIG. 1A is configured to provide fluid delivery through inner member 118, whereas FIG. 1B may be used with example inner members 118 that are not configured with fluid openings. Medical systems 100 and 200 may be used to treat a variety of conditions, including thrombosis. Thrombosis occurs when a thrombus (e.g., a blood clot or other material such as plaques or foreign bodies) forms and obstructs vasculature of a patient. For example, medical systems 100 and 200 may be used to treat deep vein thrombosis.

Medical systems 100 and 200 are configured to remove fluid via catheter 108, e.g., draw fluid from catheter 108 into discharge reservoir 104, via a suction force applied by suction source 102 to catheter 108 (e.g., to a catheter lumen of catheter 108). As detailed further below, catheter 108 includes a flexible elongated body 110 defining a catheter lumen (not shown in FIGS. 1A and 1B) and defining a distal opening 112 to the catheter lumen. Although distal opening 112 is shown at a distal-most end 108B of catheter 108, in other examples, distal opening 112 can be more proximal to the distal end 108B. For example, distal opening 112 can be a side opening defined by a side wall of catheter 108.

To treat a patient with thrombosis, a clinician may position distal opening 112 in a blood vessel of the patient near the thrombus or other occlusion, and apply a suction force (also referred to herein as suction, vacuum force, or negative pressure) to catheter 108 (e.g., to one or more lumens of the catheter) to engage the thrombus with suction force at distal opening 112. For example, suction source 102 can be configured to create a negative pressure within the catheter lumen of catheter 108 to draw a fluid, such as blood, an aspiration fluid, more solid material, or a combination thereof, into the catheter lumen via distal opening 112 of catheter 108. The negative pressure within the catheter lumen can create a pressure differential between the catheter lumen and the environment external to at least a distal portion of catheter 108 that causes fluid and other material to be introduced into the catheter lumen via distal opening 112. For example, the fluid may flow from patient vasculature, into the catheter lumen via distal opening 112, and subsequently through aspiration tubing 114 (also referred to herein as “vacuum tube 114”) into discharge reservoir 104.

Once distal opening 112 of aspiration catheter 108 has engaged the thrombus, the clinician may remove aspiration catheter 108 with the thrombus held within distal opening 112 (or appended to a distal-most end of elongated body 110 that defines distal opening 112), or suction off pieces of the thrombus (or the thrombus as a whole) until the thrombus is removed from the blood vessel of the patient, either through the catheter lumen of aspiration catheter 108 itself, and/or through the lumen of an outer catheter (or “sheath”) in which aspiration catheter 108 is at least partially positioned. The outer catheter can be, for example, a guide catheter configured to provide additional structural support to aspiration catheter 108.

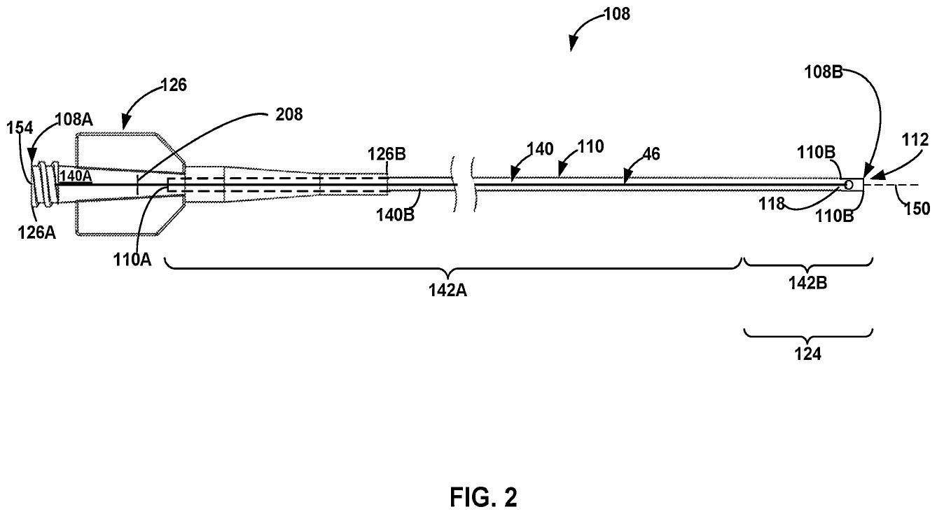

As detailed further below with respect to FIGS. 2-6, aspiration of a thrombus with an aspiration force can be performed concurrently with use of a longitudinally deployable inner member 118. Inner member 118 includes an elongated support member and a distal member configured to move proximally and distally (relative to elongated catheter body 110 and/or distal opening 112) to forcefully contact and disrupt a portion of a thrombus to facilitate aspiration of the thrombus into the catheter lumen via distal opening 112. The disruption can include, for example, macerating and/or breaking the thrombus into smaller segments and/or compressing the thrombus into a smaller volume. The distal member of inner member 118 defines a larger cross-sectional dimension than the elongated support member, the cross-section being taken in a direction transverse (e.g., orthogonal) to a longitudinal axis of the elongated support member and includes a proximal-facing surface defining a proximal taper. In some examples, the proximal taper of the distal member may extend to an outer surface of the elongated support member, and may be configured to provide a tactile response along the elongated support member when the proximal taper engages with aspiration catheter 108. For example, the proximal taper may be configured to contact a distal edge of aspiration catheter 108, e.g., at or near distal opening 112, without damaging the edge or catching on the edge and/or detaching the distal member from the elongated support member, while still providing resistance that may be felt by a clinician manipulating inner member 118.

The distal member defines a surface disruption configured to aid disruption of a thrombus. For example, the distal member may define a notch and/or a protrusion of various orientations, number, and dimension, such as one or more continuous circumferential grooves, discontinuous circumferential grooves, longitudinal grooves, continuous spiral grooves, discontinuous spiral grooves, continuous circumferential ridges, discontinuous circumferential ridges, longitudinal ridges, continuous spiral ridges, discontinuous spiral ridges, and the like, as further described herein. In some examples, the surface disruption is configured to increase the surface area of the distal member, e.g., so as to increase an amount of surface contact with a thrombus to aid in efficient disruption of the thrombus when the distal member is moved while contacting the thrombus.

As used herein, “suction force” is intended to include, within its scope, related concepts such as suction pressure, vacuum force, vacuum pressure, negative pressure, fluid flow rate, and the like. A suction force can be generated by a vacuum, e.g., by creating a partial vacuum within a sealed volume fluidically connected to a catheter, or by direct displacement of liquid in a catheter or tubing via (e.g.) a peristaltic pump, or otherwise. Accordingly, suction forces or suction as specified herein can be measured, estimated, computed, etc. without need for direct sensing or measurement of force. A “higher,” “greater,” or “larger” (or “lower,” “lesser,” or “smaller”) suction force described herein may refer to the absolute value of the negative pressure generated by the suction source on catheter 108 or another component, such as discharge reservoir 104.

In some examples, suction source 102 can comprise a pump and/or an aspiration pump (also referred to herein as “pump 102” or “vacuum source 102”). The suction source 102 can include one or more of a positive displacement pump (e.g., a peristaltic pump, a rotary pump, a reciprocating pump, or a linear pump), a direct-displacement pump (e.g., a peristaltic pump, or a lobe, vane, gear, or piston pump, or other suitable pumps of this type), a direct-acting pump (which acts directly on a liquid to be displaced or a tube containing the liquid), an indirect-acting pump (which acts indirectly on the liquid to be displaced), a centrifugal pump, and the like. An indirect-acting pump can comprise a vacuum pump, which displaces a compressible fluid (e.g., a gas such as air) from the evacuation volume (e.g., discharge reservoir 104, which can comprise a canister), generating suction force on the liquid. Accordingly, the evacuation volume (when present) can be considered part of the suction source. In some examples, suction source 102 includes a motor-driven pump, while in other examples, suction source 102 can include a syringe configured to be controlled by control circuitry 128, and mechanical elements such as linear actuators, stepper motors, and the like. As further examples, the suction source 102 could comprise a water aspiration venturi or ejector jet.

Control of suction source 102 can comprise control, operation, and the like, of any one or combination of the component(s) making up the suction source. Accordingly, in examples in which suction source 102 includes a pump and an evacuation volume, control of the suction source can comprise control of only the pump, of only the evacuation volume, or of both of those components. As in examples in which suction source 102 includes only a pump, control of suction source 102 comprises control of the pump.

Medical system 100 includes control circuitry 128 configured to control a suction force applied by suction source 102 to the catheter lumen. For example, control circuitry 128 can be configured to directly control an operation of suction source 102 to vary the suction force applied by suction source 102 to the lumen of catheter 108, e.g., by controlling the motor speed, or stroke length, volume or frequency, or other operating parameters, of suction source 102. For instance, control circuitry 128 may vary the suction force by intermittently varying the aspiration force, by periodically varying the aspiration force, or by pulsing the aspiration force, as a few non-limiting examples.

In some examples, medical system 100 of FIG. 1A is further configured to deliver fluid from fluid source 106, for example, a fluid reservoir different from discharge reservoir 104, through irrigation tubing 116 (also referred to herein as “irrigation tube 116” or “flush tube 116”) and into a lumen of inner member 118 for controlled deployment of the fluid. For example, the fluid may be delivered to reduce the amount of blood that is aspirated from the patient during the aspiration procedure. Control circuitry 128 may be configured to control communication of an aspiration fluid, such as saline, from fluid source 106 to the lumen of inner member 118. For instance, medical system 100 may include an electronic valve 120 coupled to aspiration tubing 114, irrigation tubing 116, or both. Control circuitry 128 may be operatively coupled to valve 120 to convert valve 120 between an “open” configuration and a “closed” configuration to promote or inhibit, respectively, a fluid flow through the tubing to which the valve 120 is coupled. In some such examples, medical system 100 includes a user-input mechanism enabling a user to select an amount of aspiration fluid introduced into the lumen of inner member 118 via fluid source 106 (e.g., by controlling a valve 120 coupled to irrigation tubing 116). Additionally, or alternatively, the user-input mechanism may enable the user to select an amount of fluid (including patient fluid, aspiration fluid, and/or more-viscous material, as described above) aspirated into discharge reservoir 104 (e.g., by controlling a valve 120 coupled to aspiration tubing 114). In either example, the user interface may be configured such that the respective amount of fluid may be selected or indicated in the form of a fixed amount (e.g., a measurable fluid volume), a fixed rate (e.g., a flow rate), a relative amount (e.g., a volume of “introduced” fluid relative to a volume of “aspirated” fluid, or vice versa), or a relative rate (e.g., a flow rate of introduced fluid relative to a flow rate of aspirated fluid, or vice versa).

Referring now to both medical systems 100 and 200, in some examples, inner member 118 includes a proximal actuator device (e.g., a switch, slider, knob, torque device, or the like) enabling a user, such as a clinician, to manually translate inner member 118 in proximal and distal directions relative to catheter 108. The proximal actuator device may be fixed to the inner member, or the user may be allowed to reposition the actuator device to adjust the distal member's position distal of the catheter tip. Additionally, or alternatively, control circuitry 128 can be configured to control a longitudinal movement of inner member 118, e.g., according to a predetermined motion pattern to contact, segment, and/or penetrate a portion of a thrombus that is aspirated into the catheter lumen. For instance, control circuitry 128 may be configured to actuate a longitudinal motion, a rotational motion, a motion in a direction transverse to longitudinal axis 150 (shown in FIG. 2), and/or an oscillating motion of inner member 118 within the catheter lumen of catheter 108. Control circuitry 128 may actuate the motion of inner member 118 through any suitable motion mechanism 122, such as via a rotating cam, or via linear or rotary actuator(s) which can be electrically, electromagnetically, pneumatically, or hydraulically driven to generate the desired movement of inner member 118. Such linear or rotary actuator(s) can be linear solenoid(s), rotary solenoid(s), or piezoelectric driven linear or rotary actuator(s). Regardless of the type of driving mechanism or actuator, control circuitry 128 may cause the motion mechanism 122 to actuate the motion of inner member 118 according to a single proximal or distal motion (e.g., a single motion per received user input), according to a predetermined speed or frequency, and/or according to a variable speed or frequency within a predetermined range of speeds or frequencies. As a non-limiting example, control circuitry 128 may cause inner member 118 to oscillate or otherwise periodically move at a frequency from about 5000 Hertz (Hz) to about 50,000 Hz.

Control circuitry 128, as well as other processors, processing circuitry, controllers, control circuitry, and the like, described herein, may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, control circuitry 128 may include multiple components, such as any combination of one or more microprocessors, one or more DSPs, one or more ASICs, or one or more FPGAs, as well as other discrete or integrated logic circuitry, and/or analog circuitry. In some examples, control circuitry 128 is or includes a “smart” device or system, including, but not limited to, a robotic device (e.g., a robotic surgical system), a device configured to operate with the aid of artificial intelligence (AI), a virtual reality (VR) system configured to aid a clinician with the medical procedure, cloud-based interfaces for data processing and/or data storage, or any combination thereof. In some examples, control circuitry 128 may further include, additionally or alternatively to electric-based processors, one or more controls that operate using fluid motion power (e.g., hydraulic power) in combination with or in addition to electricity. For example, control circuitry 128 can include a fluid circuit comprising a plurality of fluid passages and switches arranged and configured such that, when a fluid (e.g., a liquid or gas) flows through the passages and interacts with the switches, the fluid circuit performs the functionality of control circuitry 128 described herein.

Memory 130 may store program instructions, such as software, which may include one or more program modules, which are executable by control circuitry 128. When executed by control circuitry 128, such program instructions may cause control circuitry 128 to provide the functionality ascribed to control circuitry 128 herein. The program instructions may be embodied in software and/or firmware. Memory 130, as well as other memories described herein, may include any volatile, non-volatile, magnetic, optical, or electrical media, such as a random-access memory (RAM), read-only memory (ROM), non-volatile RAM (NVRAM), electrically erasable programmable ROM (EEPROM), flash memory, or any other digital media.

In the example shown in FIG. 1, control circuitry 128 is configured to control an amount of suction force applied by suction source 102 to the catheter lumen of catheter 108. In some examples, suction source 102 is configured to apply a substantially continuous suction force (e.g., continuous, or nearly continuous to the extent permitted by the hardware) to discharge reservoir 104, and the amount of this suction force that is transferred to the catheter lumen of catheter 108 may be adjusted by control circuitry 128. As used herein, a “continuous” suction force may include a suction force having a relative strength that is generally constant over time, or that varies in strength such that distal opening 112 experiences a constant pressure and/or a constant change in pressure to help pull thrombus portions into the catheter lumen.

In some examples, but not all examples, a distal portion of elongated body 110 of catheter 108 includes an expandable distal portion 136 configured to expand radially outward to widen distal opening 112 for engaging with a thrombus.

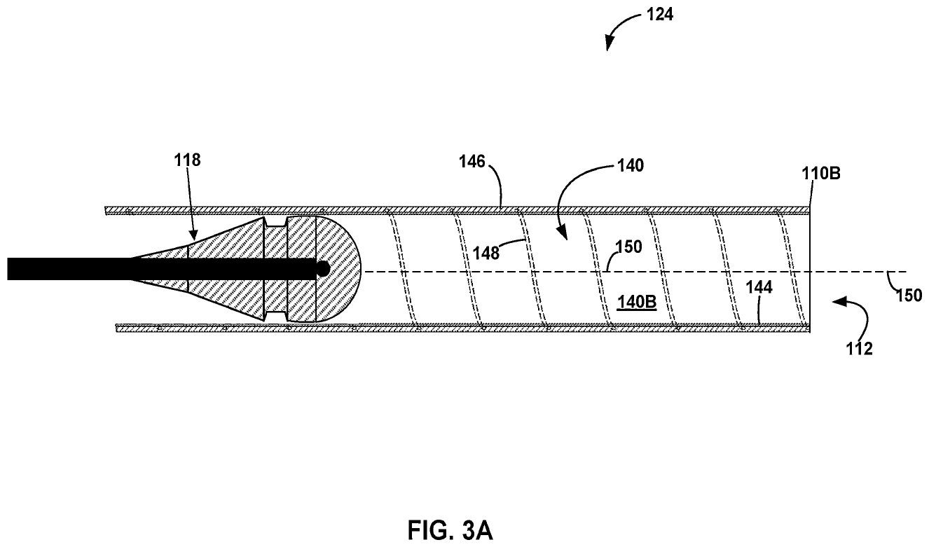

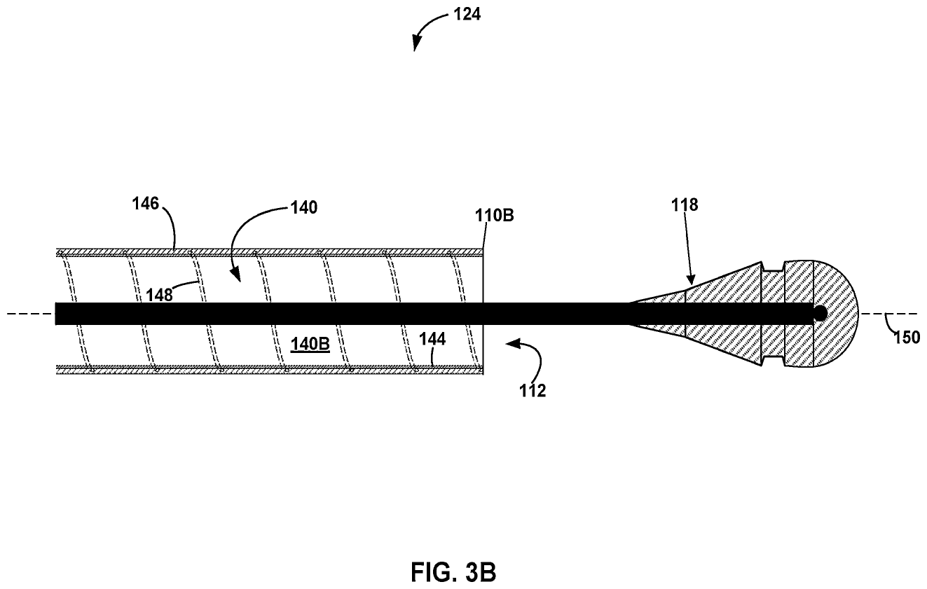

FIG. 2 is a conceptual side view of an example of aspiration catheter 108 and inner member 118 of medical system 100 of FIG. 1A and medical system 200 of FIG. 1B. FIGS. 3A and 3B are a conceptual cross-sectional views of a distal portion 124 of the example catheter 108 and inner member 118. FIG. 3A illustrates inner member 118 disposed within body lumen 140B, and FIG. 3B illustrated inner member 118 disposed within body lumen 140B and distal member 134 advanced through distal opening 112. As shown in FIG. 2 catheter 108 can include an elongated body 110 and a hub 126. Catheter 108 defines a catheter lumen 140, including a hub lumen 140A and body lumen 140B. Catheter 108 does not include expandable distal portion 136 in the example shown in FIG. 2.

Elongated body 110 is configured to be advanced through vasculature of a patient via a pushing force applied to proximal body portion 142A (e.g., via hub 126) of elongated body 110 without buckling, kinking, or otherwise undesirably deforming (e.g., ovalization). Elongated body 110 may be structurally configured to be relatively flexible, pushable, and relatively kink- and buckle-resistant, so that it may resist buckling when a pushing force is applied to a relatively proximal section of catheter 108 (e.g., via hub 126) to advance elongated body 110 distally through vasculature, and so that it may resist kinking when traversing around a tight turn in the vasculature. In some examples, elongated body 110 is configured to substantially conform to the curvature of the vasculature. Catheter 108 may be used to access tissue sites throughout the coronary and peripheral vasculature, the cranial vasculature, the gastrointestinal tract, the urethra, ureters, fallopian tubes, veins, and other hollow anatomical structures of a patient.

As shown in FIGS. 3A and 3B, in some examples, elongated body 110 includes a plurality of concentric layers, such as an inner liner 144, an outer jacket 146, and a structural support member 148 (e.g., a coil, braid, and/or hypotube) positioned between inner liner 144 and outer jacket 146. For example, structural support member 148 can be positioned between inner liner 144 and outer jacket 146 along a full length of inner liner 144 and/or outer jacket 146 or only along part of the length. Outer jacket 146, together with inner liner 144 and structural support member 148, may be configured to define elongated body 110 having the desired structural characteristics (e.g., flexibility, kink resistance, torque responsiveness, structural integrity, pushability, and column strength, which may be a measure of a maximum compressive load that can be applied to elongated body 110 without taking a permanent set). Structural support member 148 is configured to increase the structural integrity of elongated body 110 while enabling elongated body 110 to remain relatively flexible.

Structural support member 148 can include, for example, one or more braided structures, one or more coiled members defining plurality of turns, one or more hypotubes, or a combination thereof. Structural support member 148 can be made from any suitable material, such as, but not limited to, a metal (e.g., a nickel titanium alloy (Nitinol), stainless steel, tungsten, titanium, gold, platinum, palladium, tantalum, silver, or a nickel-chromium alloy, a cobalt-chromium alloy, or the like), a polymer, a fiber, or any combination thereof. Inner liner 144 may be formed using any suitable material, such as, but not limited to, polytetrafluoroethylene (PTFE), expanded PTFE (ePTFE, e.g., unidirectional ePTFE or bi-directional ePTFE), a fluoropolymer, perfluoroalkyoxy alkane (PFA), fluorinated ethylene propylene (FEP), polyolefin elastomers, Low Density Polyethylene (LDPE) (e.g., about 42D), High Density Polyethylene (HDPE), or any combination thereof. Outer jacket 146 may be formed using any suitable material including, but are not limited to, polymers, such as a polyether block amide (e.g., PEBAX®, commercially available from Arkema Group of Colombes, France), an aliphatic polyamide (e.g., Grilamid®, commercially available from EMS-Chemie of Sumter, S.C.), another thermoplastic elastomer (e.g., a thermoplastic or an elastomeric polymer), polyurethanes, polyamides, or other thermoplastic material, or combinations thereof.

In some examples, at least a portion of an outer surface of outer jacket 146 and/or inner member 118 includes one or more coatings, such as, but not limited to, an antithrombogenic coating, which may help reduce the formation of thrombi in vitro, an anti-microbial coating, a polymer layer, a polymer coating, a polymer jacket, and/or a lubricating coating.

Elongated body 110 includes a proximal body portion 142A and a distal body portion 142B, which are each longitudinal sections of elongated body 110. Elongated body 110 extends from body proximal end 110A to body distal end 110B and defines at least one body lumen 140B (also referred to as a body inner lumen). In the example shown in FIG. 2, proximal end 110A of elongated body 110 is received within a distal portion of hub 126 and is mechanically connected to hub 126 via an adhesive, welding, or another suitable technique or combination of techniques. Catheter lumen 140 of catheter 108 may be defined by portions of hub 126 and inner liner 144.

Catheter 108 may be used as an aspiration catheter to remove a thrombus or other material such as plaques or foreign bodies from vasculature of a patient. In such examples, a suction force (e.g., a vacuum) from suction source 102 may be applied to proximal end 108A of catheter 108 (e.g., via hub 126) to draw a thrombus or other blockage into catheter lumen 140.

Hub 126 may be positioned at (e.g., proximal to or at least partially overlapping with) a proximal body portion 142A of elongated body 110. Proximal end 126A of hub 126 may define catheter proximal end 108A of catheter 108 and may include a proximal opening 154 aligned with body lumen 140B of elongated body 110, such that body lumen 140B of elongated body 110 may be accessed via proximal opening 154 and, in some examples, closed via proximal opening 154. For example, hub 126 may include a luer connector, a valve, such as a hemostasis valve, or another mechanism or combination of mechanisms for connecting hub 126 to another device such as suction source 102 (FIG. 1) for performing the aspiration techniques described herein. In some examples, proximal end 108A of catheter 108 can include another structure in addition to, or instead of, hub 126.

In some examples, inner liner 144 of elongated body 110 defines at least a portion (e.g., body lumen 140B) of catheter lumen 140 of catheter 108, body lumen 140B defining a fluid passageway through elongated body 110. In some examples, body lumen 140B may extend over the entire length of inner liner 144 (e.g., from proximal end 110A of elongated body 110 to distal end 110B). Body lumen 140B may be sized to receive a medical device (e.g., another catheter, a guide member, an embolic protection device, a stent, inner member 118, a mechanical thrombectomy device, or any combination thereof), a therapeutic agent, or the like. Elongated body 110, alone or with inner liner 144 and/or other structures, may define a single catheter lumen 140, or multiple catheter lumens (e.g., two catheter lumens or three catheter lumens) of catheter 108.

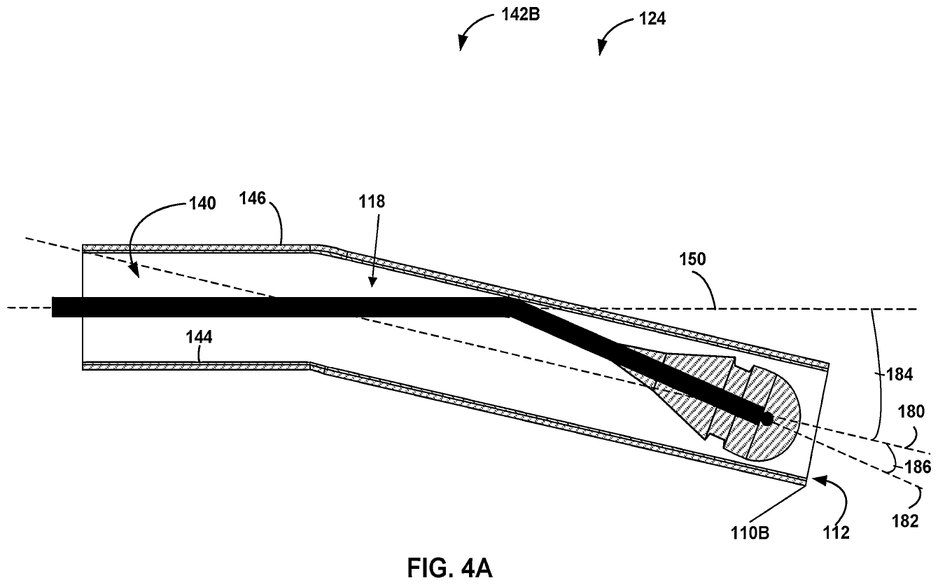

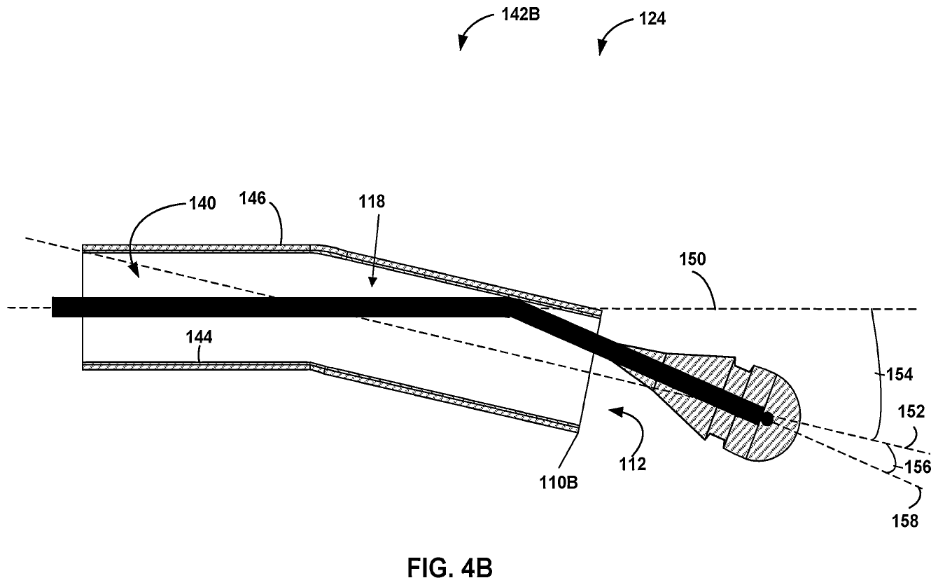

Inner member 118 may not be straight in all examples. For example, in some examples, inner member 118 is configured to be biased at an angle relative to longitudinal axis 180 of distal body portion 142B of aspiration catheter 108. FIG. 4A is a conceptual cross-sectional view of an example biased inner member 118, where the cross-section is taken through a center of biased inner member 118 and along longitudinal axes 150, 180 of catheter 108. FIG. 4B is a conceptual cross-sectional view of the example biased inner member 118 of FIG. 4A with the distal member advanced through distal opening 112. In the example shown, a distal portion of catheter 108 is angled, e.g., at angle 184 between the longitudinal axis 150 of a proximal portion of catheter 108 and longitudinal axis 180 of the angled distal portion of catheter 108. In some examples, angle 184 may be from about 10 degrees to about 30 degrees. In some examples, at least a portion of inner member 118 is biased from being substantially aligned with the longitudinal axis 180 of the distal portion of catheter 108 (and/or longitudinal axis 150 of the proximal portion of catheter 108). For example, the longitudinal axis 182 of inner member 118 and distal member 134 may be at an angle or “tilted” relative to the axis of catheter 108 near distal opening 112, namely, longitudinal axis 180 of the angled distal portion of catheter 108. In the example shown, inner member 118, and/or distal member 134, may be angled and/or biased relative to longitudinal axis 180 at bias angle 186. In some examples, bias angle 186 may be from about 0 degrees to about 30 degrees. In some examples, the bias comprises a curve, and inner member 118 may be configured to curve along with a curved distal portion 124 of catheter 108.

In some examples, aspiration catheter 108 has a curved distal portion 124, which may help improve aspiration along a vessel wall. In some examples, the bias and/or bias angle of inner member 118 may change and/or provide a tactile response to the user, e.g., a clinician may be able to determine that distal member 134 is at or near distal opening 112 and/or the distal portion of catheter 108 via a change in force and/or resistance to movement of inner member 118 due to the bias.

Inner member 118 is configured to be received (e.g., introduced and positioned) within catheter lumen 140 of aspiration catheter 108, and deployed distally outward from distal opening 112 of catheter 108 during an aspiration procedure. Inner member 118 is configured to move proximally and distally relative to elongated body 110 of catheter 108 to contact and disrupt a thrombus (e.g., break-up the thrombus into smaller pieces and/or compress the thrombus into a smaller volume) to facilitate aspiration of the thrombus into catheter lumen 140 via distal opening 112. Additionally, or alternatively, inner member 118 may be configured to move rotationally, e.g., about longitudinal axis 150, and/or in a direction transverse to longitudinal axis 150 (e.g., in a direction orthogonal to longitudinal axis 150) to disrupt the thrombus.

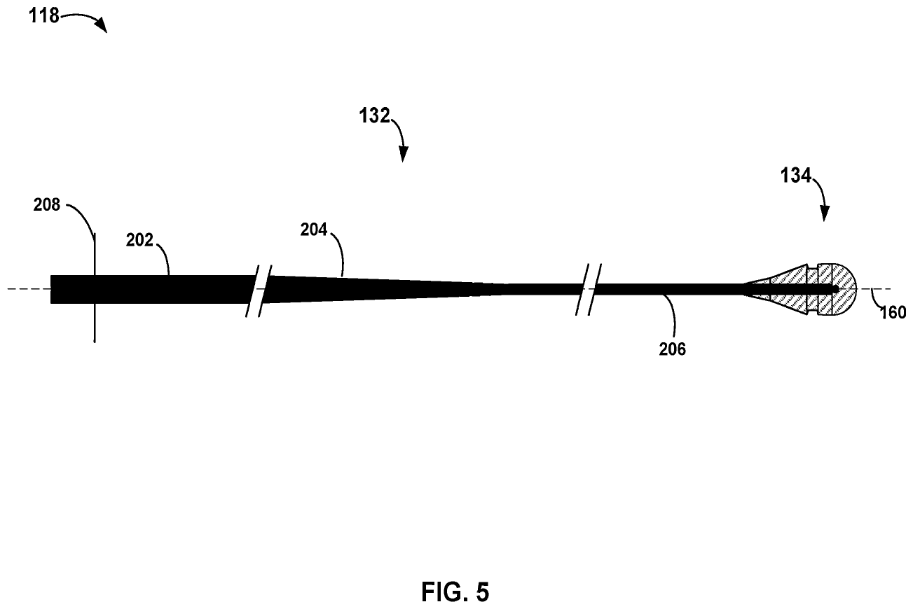

FIG. 5 is a conceptual cross-sectional view of an example inner member 118, where the cross-section is taken in a direction parallel to longitudinal axis 160. In the example shown, inner member 118 includes elongated support member 132 and distal member 134 positioned at a distal portion of elongated support member 132.

Elongated support member 132 may include any suitable elongated structure, such as, but not limited to, a solid structure or a tubular structure, such as a braided polymer shaft or a hypotube (e.g., hypotube laser-cut to have desired flexibility characteristics), defining a support member lumen 138 therein. At least a portion of distal member 134 defines a cross-sectional dimension that is larger than a cross-sectional dimension of elongated support member 132, wherein the cross-sections are taken transverse or perpendicular to longitudinal axis 160.

In some examples, elongated support member 132 has a constant maximum cross-sectional dimension (e.g., a diameter), where the cross-section is taken in a direction parallel to longitudinal axis 160. In other examples, elongated support member 132 tapers along its length, e.g., in a distal direction as shown in FIG. 5. The taper can be continuous or stepwise. In some examples, elongated support member 132 includes proximal portion 202, taper portion 204, and distal portion 206. Proximal portion 202 defines a cross-sectional dimension that is larger than a cross-sectional dimension defined by distal portion 206, and taper portion 204 defines a distal taper along at least a portion of its length that decreases from the cross-sectional dimension of proximal portion 202 to the cross-sectional dimension of distal portion 206.

The cross-sectional dimension of distal portion 206 may provide distal portion 206 with an increased flexibility relative to that of proximal portion 202. The increased flexibility may enable distal portion 206 to traverse smaller and more tortuous vasculature within the patient, e.g., within vessels that are relatively far from an insertion point of catheter 108 into the vasculature. At the same time, the cross-sectional dimension of distal portion 206 may provide kink resistance along its length, e.g., via sufficient rigidity to actuate distal member 134 to engage with a thrombus. Additionally, the cross-sectional dimensional of distal portion 206 enables a larger volume of fluid to flow a distal portion of catheter lumen 140 and a larger suction force at distal opening 112 by virtue of taking up a smaller portion of the volume within catheter lumen 140, e.g., relative to proximal portion 202.

The larger cross-sectional dimension of proximal portion 202 may provide an increased rigidity and kink resistance when manipulating inner member 118. For example, a clinician may manipulate inner member 118 in order to control movement of distal member 134 via a proximal end of proximal portion 202, and as such elongated support member 132 has to be able to support and translate mechanical motion/manipulation along its length along a tortuous path within the vasculature. As such, proximal portion 202 may be configured with a rigidity and/or stiffness that is relatively higher than distal portion 206 in order to translate a force applied at a proximal end of proximal portion 202 to distal member 134 attached to a distal end of distal portion 206.

Taper portion 204 transitions the cross-sectional dimension of elongated support member 132 from that of proximal portion 202 to distal portion 206. At the same time, taper portion 204 is configured to provide sufficient rigidity for kink resistance and to translate forces applied to proximal portion 202 along its length to distal portion 206, and to provide sufficient flexibility to traverse a potentially increasingly tortuous vasculature of the patient.

In some examples, inner member 118 and catheter 108 are configured to limit the extent to which inner member 118 can be distally advanced out of distal opening 112 of catheter 108. This may, for example, help reduce the possibility of inner member 118 advancing into regions of vasculature that should be avoided and which may not benefit from the presence of inner member 118. In the example shown in FIG. 5, for example, proximal portion 202 of elongated support member 132 includes limit member 208. Limit member 208 is configured to interact with proximal end 108A (FIG. 2) of catheter 108 or a protrusion in catheter lumen 140 to prevent further distal movement of inner member 118. In the example shown in FIG. 2, limit member 208 is configured to engage with body proximal end 108A of catheter 108 to help prevent inner member 118 from moving further in a distal direction. In some examples, limit member 208 may be a tab attached to and/or integral with elongated support member 132. The tab can extend radially outward from longitudinal axis 160 in any suitable directions, e.g., all the way around the outer perimeter of elongated support member 132 or only partially around the outer perimeter.

In some examples, distal member 134 is formed from a substantially rigid material, e.g., firm and/or non-compliant material, which is configured to resist deformation during contact with a thrombus. For example, in contrast to an expandable balloon or another expandable structure, distal member 134 may have a fixed size. Thus, in some examples, distal member 134 is configured to remain relatively the same size in catheter lumen 140, as well as outside of catheter lumen 140. While distal member 134 may be formed from a material with some elasticity, such that distal member 134 may compress slightly while in catheter lumen 140 in some examples, distal member 134 is configured to maintain generally the same maximum cross-sectional dimension (e.g., a diameter) in a direction orthogonal to longitudinal axis 160, in catheter lumen 140 and when deployed out distal opening 112 of catheter 108. For example, a maximum cross-sectional dimension of distal member 134 when in catheter lumen 140 can be within 10%, within 5% or even within 1% of the maximum cross-sectional dimension when no external compressive forces are being applied to distal member 134 by catheter 108 or the like. In some examples, distal member 134 is formed from material having a hardness of 25D to 45D, or from 63D to 72D, or from 25D to 75D on the Shore D hardness scale.

In other examples, distal member 134 is formed from a substantially elastic material, e.g., a compliant material such as an expandable balloon or another expandable structure. This, in some examples, distal member 134 is configured to have an unexpanded size, e.g., within in catheter lumen 140, as well as and expanded size, e.g., outside of catheter lumen 140. In some examples, distal member 134 may be formed from a composite of rigid and elastic materials. In some examples, distal member 134 may be formed from a material configured to have shape memory.

In the example shown, distal member 134 comprises a bulb, e.g., a bulbous shape, including an atraumatic convex distal surface profile. Distal member 134 is attached to and/or integral with a distal end of distal portion 206, and inner member 118 terminates at a distal end of distal member 134. For example, inner member 118 and/or distal member 134 do not include an elongated element, guidewire, or any other structure extending distally from distal member 134. Distal member 134 is configured to be atraumatic to a vessel wall, and is devoid of distal structures configured to penetrate through a thrombus that may also be traumatic to a vessel.

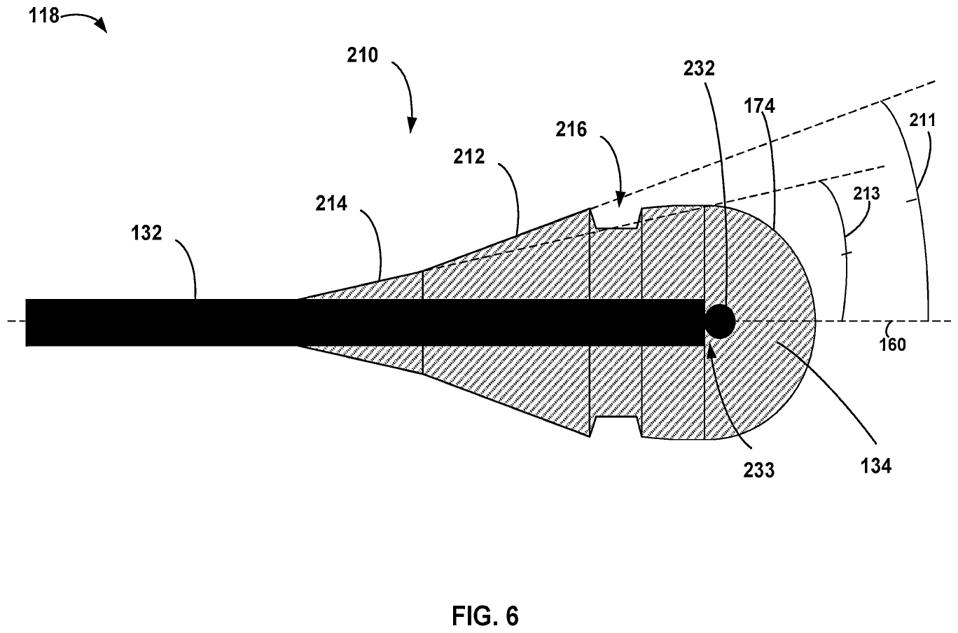

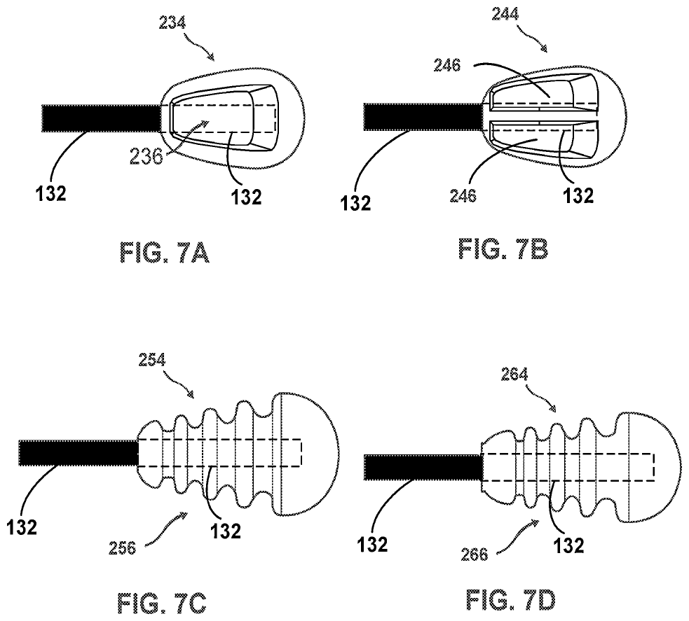

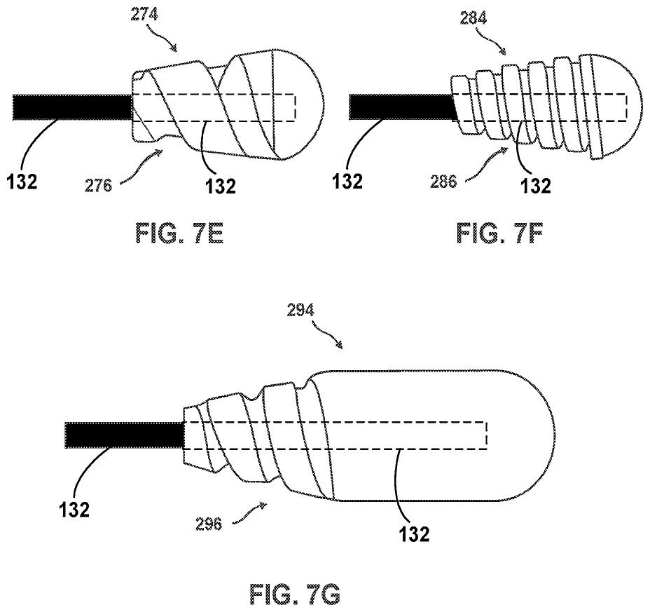

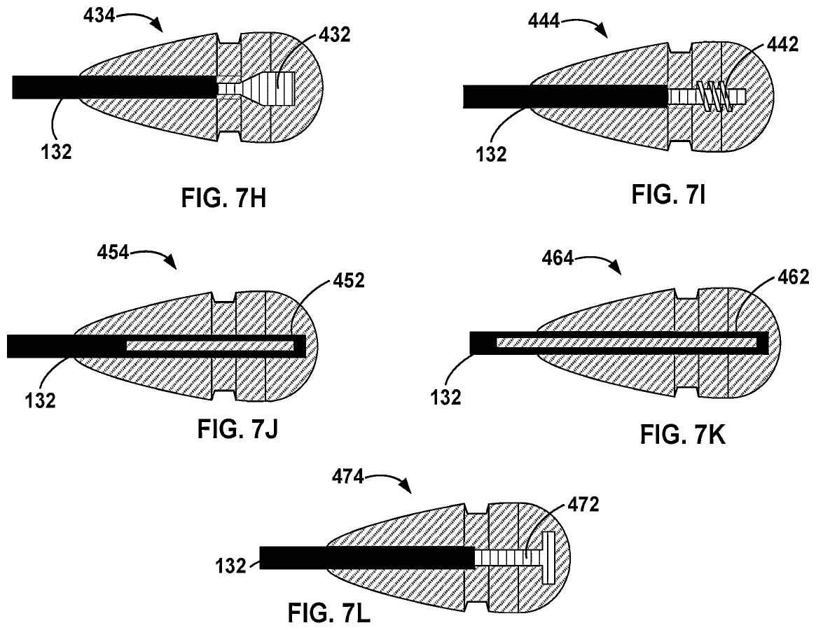

FIGS. 6-7L are conceptual illustrations of various example distal members 134, 234, 244, 254, 264, 274, 284, 294, 434, 444, 454, 464, and 474 respectively, of inner member 118. FIG. 6 is a conceptual cross-sectional view of an example distal member 134 of inner member 118. FIGS. 7A-7G illustrate other example distal members with other sizes, shapes, and surface disruptions. FIGS. 7H-7L illustrate other example distal members with other retention members, e.g., retention members 432, 442, 452, 462 and 472. FIGS. 7A-7L also illustrate the extent to which elongated support member 132 can be co-extensive with distal members. For example, elongated support member 132 can extend relatively close to a distal-most end of distal member 134, or at least over a majority of a length of distal member 134, the length being measured a longitudinal axis of inner member 118. In other examples, a distal-most end of elongated support member 132 can terminate at or proximal to a midpoint of distal member 134, the midpoint between mid-way between the proximal and distal ends of distal member 134.

In the example shown in FIG. 6, inner member 118 includes elongated support member 132, retention member 232, and distal member 134. Distal member 134 defines proximal taper 210. In some examples, an outer surface of distal member 134 along proximal taper 210 has an angle 211 with reference to longitudinal axis 160 of elongated support member 132 of about 10 degrees to 30 degrees. Proximal taper 210 tapers from a larger outer perimeter section of distal member 134 towards an outer surface of elongated support member 132 in a proximal direction and can have any suitable configuration. In the example shown, proximal taper 210 comprises a first taper portion 212 having a first taper angle 211 measured between an outer surface of distal member 134 and longitudinal axis 160 and a second taper portion 214 proximal to first taper portion 212, wherein the second taper portion has a second taper angle 213 measured between an outer surface of distal member 134 and longitudinal axis 160, the second taper angle 213 being less than the first taper angle 211. For example, first taper portion 212 may have a 20-degree angle, a 30-degree angle, a 40-degree angle, or any suitable angle relative to the longitudinal axis. Second taper portion 214 may have a 5-degree angle, a 10-degree angle, a 15-degree angle, or any suitable angle relative to the longitudinal axis. In some examples, proximal taper 210 does not include first and second taper portions 212, 214 and may be a single portion having a taper angle of 5 degrees to 40 degrees, such as about 15 degrees to 20 degrees, or 10 degrees to 30 degrees, or any other suitable taper angle.

In some examples, any or all of proximal taper 210, first taper portion 212, and/or second taper portion 214 may define a plurality of angles and/or a curve. For example, any or all of proximal taper 210, first taper portion 212 and/or second taper portion 214 may define a convex and/or concave curve including angles from 0 degrees to 5 degrees, from 0 degrees to 20 degrees, from 0 degrees to 40 degree, or from 0 degrees to any suitable angle.

In some examples, proximal taper 210 is configured to provide a tactile response to a clinician during use of inner member 118 with catheter 108. For example, proximal taper 210 may be configured to provide a tactile response along elongated support member 132 to a clinician and/or user manipulating inner member 118 at a proximal end of inner member 118. As an example, proximal taper 210 may provide a tactile response when proximal taper engages with aspiration catheter 108, e.g., at distal opening 112. For example, a clinician may move inner member 118 distally and proximally through a thrombus while aspirating the thrombus via catheter 108. When moving proximally (e.g., drawing distal member 134 back towards distal opening 112), proximal taper 210 may engage with an edge and/or lumen inner sidewall of catheter 108. In addition, proximal taper 210 may guide distal member 134 along distal opening 112 and into catheter lumen 140, such as by centering distal member 134 with distal opening 112 as the clinician proximally withdraws distal member 134 through distal opening 112. This gradual centering of distal member 134 and withdrawal into catheter lumen 140 may help and prevent distal member 134 from catching and/or being stopped by an edge catheter 108. In other words, proximal taper 210 may prevent distal member 134 from catching on, and being detached from elongated support member 132 by, a distal edge of catheter 108 when being moved proximally. At the same time, proximal taper 210 may have an angle sufficient to cause a resistance when contacting catheter 108, either at a distal edge of catheter 108 at distal opening 112 or along an inner sidewall of catheter lumen 140. The clinician may feel the resistance, and may determine the position of distal member 134 to be at or near distal opening 112 based on the resistance/tactile feel via elongated support member 132.

In some examples, including examples in which proximal taper 210 may not contact catheter 108, proximal taper 210 may have an angle configured to cause a tactile response comprising a resistance and/or suction force via the suction force of aspiration catheter 108. In other words, the clinician may determine the position of distal member 134 based on changes to a suction force/resistance at distal member 134 approaches distal opening 112 and affects the flow of the fluid into catheter lumen 140. The changes to the suction force may be perceived by the clinician tactically or visually based on an amount of material (e.g., fluid) drawn through catheter lumen 140 and into discharge reservoir 104 (FIGS. 1A and 1B). In some examples, proximal taper 210 is configured to reduce a turbulent flow of the fluid when distal member 134 is at or near distal opening 112, e.g., so as to reduce and/or prevent portions of the thrombus from being forced out of and/or past distal opening 112 via turbulent fluid flow.

In some examples, the outer maximum cross-sectional dimension (e.g., an outer diameter) of distal member 134 is configured to, along with the inner cross-sectional dimension of catheter lumen 140, compress a thrombus. For example, if the outer diameter of distal member 134 is too small, a thrombus may not be sufficiently compressed, e.g., between distal member 134 and the inner wall and/or distal end 108B of catheter 108 or an inner surface of catheter 108 defining catheter lumen 140. In some examples, the outer cross-sectional diameter of distal member 134 is from 80% to 95% the inner cross-section diameter of catheter lumen 140.

In some examples, distal member 134 includes surface disruption 216. In the example shown, surface disruption 216 is a continuous circumferential groove, recess, and/or ring, where the circumferential direction extends about longitudinal axis 160. In some examples, surface disruption 216 may be located only along proximal taper 210. In other examples, surface disruption 216 may be located at a portion of distal member 134 in addition to or other than along proximal taper 210. In some examples, the surface disruption 216 may be located at or define a transition region between the proximal taper 210 and a distal portion of the distal member 134, which may have a substantially constant diameter. In some examples, surface disruption 216 is configured to increase a surface area of distal member 134, e.g., for engagement with the thrombus. In some examples, surface disruption 216 is configured to disrupt, macerate, and/or compact the thrombus, e.g., by providing one or more edges in the surface of distal member 134 that may traverse through the thrombus to disrupt the thrombus, interact with catheter 108 to shear and/or compress the thrombus, or the like.

In other examples, surface disruption 216 can have other configurations in addition to or instead of the circumferential groove, recess, and/or ring shown in FIG. 6. For example, FIGS. 7A-7G illustrate conceptual side views of example distal members 234-294, which may be substantially similar to distal member 134, but have different sizes, surface disruptions, and/or proximal taper 210 angles. FIG. 7A illustrates an example distal member 234 includes surface disruption 236 comprising a notch, which can be an absence of material (e.g., as opposed to a protrusion) and an indentation into an outer surface of distal member 234. FIG. 7B illustrates an example distal member 244 including surface disruption 246 comprising a plurality of notches distributed about the outer perimeter (e.g., circumference) of distal member 244, e.g., two notches that are visible in FIG. 7B and two that are on the non-visible side in the example shown. In some examples, distal member 244 may have any number of notches, for example, two or three notches, or five or more notches, and the one or more notches can have any suitable size. In some examples, surface disruptions 236 and 246 may comprise a protrusion (not shown) rather than a notch. For example, distal member 234 may comprise a single protrusion, and distal member 234 may comprise a plurality of protrusions. In some examples, surface disruption 236 may comprise both a notch and a protrusion, e.g., at different positions on the surface of distal member 234, and surface disruption 246 may comprise a plurality of both notches and protrusions in any combination.

As shown in FIGS. 7C and 7D, distal members 254 and 264 include surface disruptions 256 and 266, respectively, which comprise a plurality of continuous circumferential grooves (e.g., or recesses, or rings), which are longitudinally spaced apart from each other along a length of the respective distal member 254, 264. Surface disruptions 256 and 266 each include four grooves, however, in some examples, surface disruptions 256 and 266 may comprise more or fewer grooves, e.g., one, two, or three grooves, or five or more grooves. In some examples, surface disruptions 256 and 266 may comprise ridges (e.g., continuous circumferential protrusions, not shown in FIGS. 7C and 7D), rather than grooves. In some examples, surface disruptions 256 and 266 may comprise one or more of both grooves and ridges in any combination.

As shown in FIGS. 7E and 7F, distal members 274 and 284 include surface disruptions 276 and 286, respectively, which comprise a plurality of continuous spiral grooves (e.g., recesses or rings), which extend both longitudinally and around an outer perimeter (e.g., circumferentially) of the respective distal member 274, 284. Surface disruptions 276 and 286 are examples of spiral grooves having different widths and spiral angles. Surface disruptions 276 and 286 may comprise spiral grooves having any suitable widths and spiral angles. In some examples, surface disruptions 276 and 286 may comprise ridges (e.g., continuous spiral protrusions, not shown in FIGS. 7E and 7F), rather than spiral grooves. In some examples, surface disruptions 276 and 286 may comprise one or more of both spiral grooves and ridges in any combination, e.g., of the same or differing widths and spiral angles which may or may not overlap and form a surface disruption pattern.

FIG. 7G illustrates distal members 294 which includes surface disruption 296 which comprises a plurality of continuous spiral grooves (e.g., or recesses, or rings) and an extended exterior surface 272. Surface disruption 296 may be substantially similar to surface disruptions 276 and 286. While only shown in FIG. 7G, the extended exterior surface 272 may be incorporated into any other disclosed distal member.

In some examples, surface disruption 216 may comprise any of the surface disruptions 236-296 described above, or any other suitable surface disruption. For example, surface disruption 216 may comprise one or more discontinuous circumferential grooves that extend around an outer perimeter of the respective distal member, one or more continuous or discontinuous longitudinal grooves that extend parallel to or nearly parallel (e.g. within 5 to 10 degrees of) longitudinal axis 160, one or more discontinuous spiral grooves that extend both longitudinally and an around an outer perimeter of the respective distal member, one or more discontinuous circumferential ridges, one or more longitudinal ridges, one or more discontinuous spiral ridges, or the like.

Referring now to FIG. 6, in some examples, inner member 118 includes retention member 232 configured to aid attachment between the elongated support member 132 and distal member 134. In some examples, retention member 232 is configured to increase a surface area of a part of elongated support member 132 that attaches to the distal member 134. For example, retention member 232 may be enlarged in a radial direction relative to elongated support member 132, e.g., such that a maximum cross-sectional dimension of retention member (in a direction transverse to longitudinal axis 160) is greater than a cross-sectional dimension of elongated support member 132 at the part of support member 132 immediately adjacent to retention member 232. In some examples, retention member 232 is integral with elongated support member 132. In other examples, retention member 232 is attached to a distal end and/or portion of elongated support member 132. In some examples, retention member 232 is welded to the distal end of elongated support member 132.

In some examples, retention member 232 is a distal ball, e.g., at a distal-most end of support member 132 or near a distal-most end of support member 132, and distal member 134 is a polymer and/or elastomer configured to surround and attach to the distal ball. In other example, retention member 232 is a coil. In some examples, at least a portion of distal member 134 is configured to be formed proximal to at least a portion of retention member 232, e.g., such as at volume 233 of FIG. 6. For example, a portion of distal member 134 formed to surround and be proximal to at least a portion of retention member 232 may increase an attachment force of distal member 134 to elongated support member 132, e.g., to aid in preventing distal member 134 from detaching from elongated support member 132 such as when elongated support member 132 is moved in the proximal direction and encounters resistance, e.g., from catheter 108 and/or ancillary devices. The distal ball extends radially away from an outer surface of elongated support member 132. In some examples, the distal ball has a diameter that is from 1.5 to 3 times that of the diameter of the elongated support member (the diameters being measured in a direction perpendicular to longitudinal axis 160). In other examples, the distal ball has a cross-sectional dimension that is less than a cross-sectional dimension of elongated support member 132, e.g., such that it is an indentation in elongated support member 132.

In other examples, retention member 232 can have other configurations in addition to or instead of the distal ball shown in FIG. 6. For example, FIGS. 7H-7L are schematic cross-sectional views of example distal members 434, 444, 454, 464, 474 including retention members 432, 442, 452, 462, and 472, respectively, which may be substantially similar to retention member 232, but have different sizes, shapes, and/or features. The cross-sections shown in FIGS. 7H-7L are taken through a central longitudinal axis of elongated support member 132. FIG. 7H illustrates an example distal member 434 including retention member 432 comprising a profiled wire, which may have spade-like shape in cross-section, the cross-section being taken parallel to a longitudinal axis of distal member 434. FIG. 7I illustrates an example distal member 444 including retention member 442 comprising a helix, spiral, or coil anchor. The spade, helix, spiral, or coil configuration of retention member 432, 442 exhibits a relatively high surface area for a given length (measured along a longitudinal axis of the device), which increases the surface area connected to the material of distal member 434, 444. This increased surface area may help increase the retention force attaching the respective distal member 432, 442 to elongated support member 132.

FIG. 7J illustrates an example distal member 454 including retention member 452 comprising a slotted wire. For example, retention member 452 may comprise a slot formed in a distal portion of elongated support member 132 within distal member 444. In some examples, the material of distal member 454 fills the slot and increase the retention force attaching distal member 454 to elongated support member 132. FIG. 7K illustrates an example distal member 464 including retention member 462 comprising another example slotted wire similar to retention member 452 of FIG. 7J, but with the slot extending proximally beyond distal member 464. In some examples, the slot of retention member 462 further increases the amount of material of distal member 464 filling and adhering to retention member 462, e.g., relative to distal member 454 and retention member 452, thereby further increasing the retention force attaching distal member 464 to elongated support member 132.

FIG. 7L illustrates an example distal member 474 including retention member 472 comprising a T-weld, which may comprise a washer T-weld, a wire T-weld, or a combination washer and wire T-weld. The T-shape configuration of retention member 472 helps to increase a surface area of retention member 472 for a given length, which increases the surface area connected to the material of distal member 474. This increased surface area may help increase the retention force attaching the respective distal member 474 to elongated support member 132.

Referring now to FIG. 6, in some examples, elongated support member 132 and distal member 134 are formed from different materials, which may not directly attach to each other very well. For example, elongated support member 132 may be formed from a metal (e.g., Nitinol) and distal member 134 may be formed from a polymer. To help facilitate the mechanical connection between elongated support member 132 and distal member 134, inner member 118 can include a material between elongated support member 132 and distal member 134 that helps secure the mechanical connection therebetween. For example, in some examples, an outer surface of elongated support member 132 includes a polymer and/or elastomer layer configured to increase an adhesion between distal member 134 and the outer surface of elongated support member 132. As an example, distal member 134 may be a polymer and/or elastomer molded and/or formed over and surrounding a distal portion of elongated support member 132, and elongated support member 132 includes a polymer and/or elastomer layer. In some examples, the polymer and/or elastomer of distal member 134 may be configured to match, bond, and/or adhere to a polymer and/or elastomer layer disposed and/or formed over a distal portion of elongated support member 132, e.g., to further increase retention of distal member 134 to elongated support member 132 and/or retention member 232.

In some examples, the polymer layer may comprise a polymer coating and/or a tubular polymer jacket positioned over an outer surface of elongated support member 132. In some examples, distal member 134 may be formed with a polymer and/or elastomer configured to have dimensional stability when injection molded and to be compatible with the jacket polymer and/or elastomer disposed, coated, and/or formed over a distal portion of elongated support member and/or retention member 232, e.g., at least a portion of distal member 134 and/or the polymer jacket may be Pebax.® In some examples, distal member 134 may include a radiopaque material, e.g., tungsten, platinum, platinum iridium, barium sulfate, or any combination thereof, to enable a clinician to determine the position of distal member 134, e.g., relative to distal end 108B of catheter 108, in medical imaging. As an example, distal member 134 may be formed at least partially from a tungsten loaded block copolymer including rigid polyamide blocks and soft polyether blocks (e.g., Pebax®).

In some examples, proximal taper 210 is configured to increase a tensile strength of distal member 134 and/or the attachment and/or adhesion of distal member 134 to elongated support member 132. For example, proximal taper 210 may extend the length of distal member 134 and thereby provide additional surface area of distal member 134 to bond and/or adhere to elongated support member 132, e.g., a larger surface area of a polymer/elastomer distal member 134 to bond with a polymer/elastomer jacket of elongated support member 132.

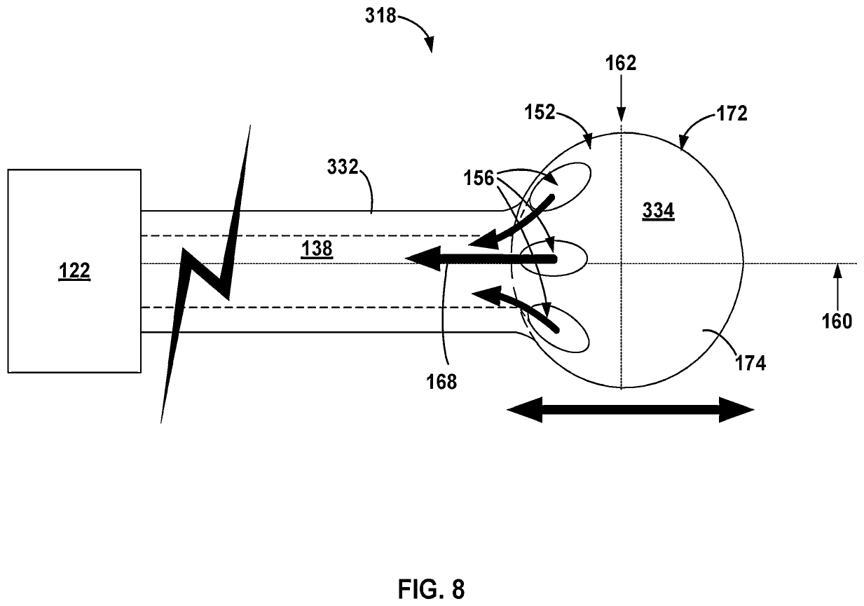

FIG. 8 is a conceptual side view of another example inner member 318. In the example shown, inner member 318 includes elongated support member 332 and distal member 334 positioned at a distal portion of elongated support member 332. Inner member 318, elongated support member 332, and distal member 334 may be substantially similar to inner member 118, elongated support member 132, and distal member 134 described above except that inner member 318 is configured to deliver a fluid.

Distal member 334 includes an exterior surface 172 (also referred to herein as an outer surface) which, in some examples, may include an antithrombogenic coating, a lubricious coating, a polymer coating, and/or one or more surface textures configured to help disrupt a thrombus. Exterior surface 172 includes proximal-facing surface 152, e.g., the portion of the exterior surface 172 facing catheter distal opening 112 when distal member 334 is deployed from distal opening 112 and when at least part of support structure 332 is positioned within catheter lumen 140. For example, in examples in which distal member 334 is generally spherical (e.g., spherical, or nearly spherical to the extent permitted by manufacturing tolerances), proximal-facing surface 152 is the part of exterior surface 172 proximal to hemispherical plane 162.

In some examples, proximal-facing surface 152 defines one or more openings 156 defining respective fluid pathways between support member lumen 138 and the exterior environment. In some examples, the only fluid-delivery openings defined by distal member 334 are on proximal-facing surface 152 and not on distal-facing surface (on an opposite side of hemispherical plane 162 from proximal-facing surface 152 in the example shown in FIG. 4). By confining openings 156 to proximal-facing surface 152, inner member 318 is configured to direct a flow of a surgical fluid, such as saline, in a generally proximal direction in order to augment an applied proximal aspiration force. In addition, by confining openings 156 to proximal-facing surface 152 or confining fluid delivery in only the proximal direction, the fluid can direct a thrombus or thrombus segments towards catheter lumen 140 and suction force applied to catheter lumen 140 may help further draw the thrombus or thrombus segments into catheter lumen 140. In contrast, fluid flow in a distal direction (in a direction away from catheter lumen 140) may cause a thrombus or thrombus segments in a direction away from catheter lumen 140, which may result in a more inefficient (e.g., longer) medical aspiration procedure.

As described above, a proximal fluid flow provided by openings 156 may provide an additional benefit by diluting and/or displacing an amount of patient fluid, such as blood, that might otherwise be incidentally aspirated during the procedure. For instance, the proximal-facing orientations of openings 156 are configured to cause the proximal fluid flow to be directed proximally, such that the fluid displaces and/or dilutes a volume of the patient's blood before it is aspirated through distal opening 112, into catheter lumen 140, and into discharge reservoir 104. Reducing incidental patient blood withdrawal in this manner is believed to improve patient outcomes, such as by reducing patient recovery time, and the like. In addition, in some cases, directing fluid in the proximal direction and not in a distal direction may also be beneficial because fluid delivered in a distal direction may have an intended effect of diverting material intended for aspiration away from distal opening 112 of catheter 108, and may in some cases interfere with efficient aspiration. However, as discussed above, in some examples, inner member 318 is configured to selectively enable fluid flow in a distal direction.

Openings 156 can be distributed along distal member 334 in any suitable matter, such as evenly distributed about an outer perimeter of distal member 334 (and in a direction extending about longitudinal axis 160) or unevenly distributed about the outer perimeter and/or longitudinally aligned along longitudinal axis 160 or having different longitudinal positions from at least one other opening 156.

Openings 156 may define any suitable geometric shape, including circular, oval, a quadrilateral shape, a tear drop shape (including sides tapering in a proximal or distal direction), an hourglass shape, or another suitable geometric shape. In some examples, openings 156 may also be configured or positioned along distal member 334 to direct a fluid flow 168 in a particular direction. For instance, openings 156 may be oriented to direct the fluid flow proximally, e.g., along a line parallel to longitudinal axis 160 or within about 90 degrees of longitudinal axis 160. In other examples, openings 156 may be oriented so as to direct the fluid flow both proximally and radially inward or outward, as desired.

In addition to proximal-facing openings 156, in some examples, a distal-facing surface 174 of distal member 334 may include one or more distal-facing openings (not shown). The distal-facing openings can be configured similarly to proximal-facing openings 156 in some examples, in shape, size, and/or distribution about distal member 334.

In some examples, inner member 318 is configured to enable a user to selectively deliver fluid to one or more proximal-facing openings 156 (e.g., a subset of openings 156 or all of the openings) and, if present, to one or more distal-facing openings (e.g., a subset of the distal-facing openings or all of the distal-facing openings). For instance, a subset (e.g., one or more, but not all) or all of proximal-facing openings 156 can be fluidically coupled to a common fluid delivery lumen within elongated support member 332 and, if inner member 318 includes one or more distal-facing openings, one or more a subset (e.g., one or more, but not all) or all of the distal-facing openings 156 can be fluidically coupled to a different fluid delivery lumen within elongated support member 332.

Elongated support member 332 can include any suitable number of lumens for the fluid delivery to one or more subsets of proximal-facing openings 156 and one or more subsets of the distal-facing openings. For example, in some examples, all of proximal-facing openings 156 are fluidically coupled to the same fluid delivery lumen. In other examples, a first subset of proximal-facing openings 156 are fluidically coupled to a first fluid delivery lumen and a second subset of proximal-facing openings 156 different from the first subset are fluidically coupled to a second fluid delivery lumen fluidically isolated from the first fluid delivery lumen. Similarly, if present, in all of the distal-facing openings can be fluidically coupled to the same fluid delivery lumen or a first subset of distal-facing openings can be fluidically coupled to one fluid delivery lumen and a second subset of distal-facing openings different from the first subset are fluidically coupled to a different fluid delivery lumen.

In examples in which elongated support member 332 includes multiple fluid delivery lumens to enable selective delivery to different openings, medical system 100 includes an actuator (e.g., provided by a user interface coupled to control circuitry 128 or a switch) configured to enable the user to select the subset of fluid delivery openings for fluid delivery. For example, in response to receiving user input via the user interface, control circuitry 128 can actuate a valve (e.g., a multiple-way valve) to fluidically coupled fluid source 106 with the one or more lumens of support structure 332 corresponding to the openings indicated by the user input. As another example, a user may manually move a switch to fluidically couple fluid source 106 with the one or more lumens of support structure 332 corresponding to the fluid delivery openings to be used for fluid delivery. For example, the actuator can enable a user (e.g., a clinician) to select or alternate between a proximal-directed fluid flow from proximal-facing openings 156 and a distal-directed fluid flow from the distal-facing openings, to provide more control over the aspiration procedure. As another example, the actuator can enable a user to select only a subset of proximal-facing openings 156 or only a subset of distal facing openings for fluid delivery.

In the example shown in FIG. 8, distal member 334 defines a substantially spherical shape. However, this example configuration of distal member 334 is not intended to be limiting, and FIGS. 5-7E include other example configurations. Inner member 318 can have any suitable configuration for engaging and segmenting a thrombus within a patient's vasculature. For instance, in other examples, inner member 318 may include an oblong or “egg” shape any of the shapes described with reference to FIGS. 5-7E, or any other suitable geometric three-dimensional shape. In addition, distal member 334 may include any of the surface disruption features disclosed herein.

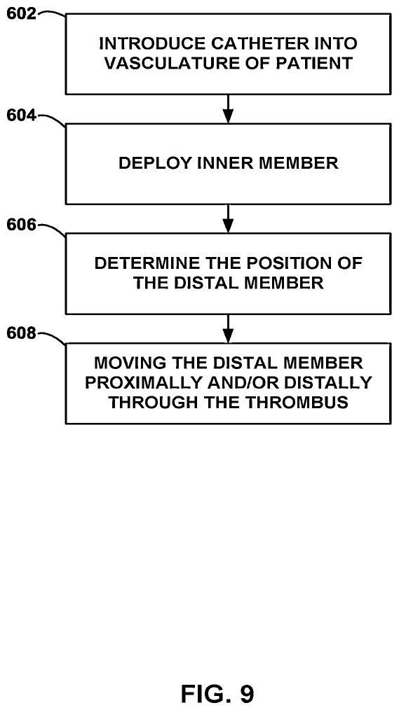

FIG. 9 is a flow diagram of an example method of using catheter 108 of FIGS. 2 and 3. The example technique of FIG. 9 is described with respect to medical systems 100 and 200, catheter 108, and inner member 118 of FIGS. 1-6, the example technique of FIG. 9 may be performed using any system including an aspiration catheter and inner member described herein. The technique of FIG. 9 may be performed by any suitable user, such as a clinician, and the like.

A clinician may introduce aspiration catheter 108 into vasculature of a patient and distally advance aspiration catheter 108 toward a thrombus within the vasculature of the patient (602). For example, the clinician may initially introduce a guidewire, guide catheter, or another guide member into the vasculature of the patient to a target treatment site. Elongated body 110 may then be introduced over the guidewire and advanced to the target treatment site. Additionally, or alternatively, the clinician may advance catheter 108 into vasculature of a patient with the aid of a guide catheter or sheath. For example, the clinician may initially introduce a guide catheter into vasculature of a patient and position the guide catheter adjacent a target treatment site. Aspiration catheter 108 may then be introduced through an inner lumen of the guide catheter.

The clinician may deploy distal member 134 of inner member 118 from distal opening 112 of aspiration catheter 108 and into contact with a thrombus (604). In some examples, the clinician may position inner member 118 within catheter lumen 140 as aspiration catheter 108 is navigated to the target treatment site, and deploy distal member 134 out of distal opening 112 from catheter lumen 140 by moving elongated support member 132. In other examples, the clinician may introduce inner member 118 into catheter lumen 140 and navigate inner member 118 to the target treatment site through catheter lumen 140 after aspiration catheter 108 is navigated to the target treatment site. Additionally, or alternatively, the clinician may advance a guidewire to the target treatment site and advance inner member 118 to the treatment site along the guidewire (e.g., with the guidewire positioned inside an inner lumen of inner member 118).

During the medical procedure (and not necessarily in the order shown in FIG. 6), the clinician may determine a position of distal member 134 based on a tactile response along elongated support member 132 (606). For example, the clinician may feel a tactile response via elongated support member 132, such as a resistance to movement and determine that distal member 134 is proximate the distal end of aspiration catheter 108. In some examples, the clinician may feel a tactile response via elongated support member 132 corresponding to proximal taper 210 engaging with aspiration catheter 108. In some examples, the clinician may distally move distal member 134 to increase a suction force of aspiration catheter 108 based on determining that the position of distal member 134 is proximate the distal end of aspiration catheter 108, e.g., to move distal member 134 out and away from distal opening 112 to clear catheter lumen 140 of distal member 134 and allow a greater suction force to be applied at distal opening 112, e.g., to suction a thrombus into catheter lumen 140.

In some examples, the clinician may move distal member 134 with the aid of medical imaging, e.g., distal member 134 may include a radiopaque material that may be used to indicate its position via medical imaging. In other examples, the clinician may move distal member 134 without the aid of medical imaging, e.g., the clinician may determine the position of distal member 134 based on the tactile response. For example, a tactile response provided by proximal taper 210 of distal member 134 may allow the clinician to determine the position of the distal member 134 and to move distal member 134 without the aid of medical imaging and thereby reduce the amount of radiation that the patient is exposed to.

The clinician moves distal member 134 proximally and/or distally relative to catheter 108 to contact and disrupt the thrombus, e.g., to break the thrombus into smaller portions, to compress the thrombus or thrombus portions into smaller volumes, and/or to clear or unclog catheter lumen 140 of the thrombus (608). For example, the clinician may repeatedly move distal member 134 distally and proximally relative to catheter 108. In other examples, the clinician may move distal member 134 in other directions, e.g., by rotating distal member 134 relative to catheter 108, by moving distal member 134 in a radial direction relative to a central longitudinal axis of catheter 108 and/or catheter lumen 140, oscillating distal member 134, or the like. In some examples, the clinician may advance distal member 134 through the thrombus, and withdraw distal member 134 back into the thrombus thereby compressing the thrombus between an outer surface of distal member 134 and an inner surface of catheter lumen 140.

The clinician may cause suction source 102 (FIGS. 1A and 1B) to apply a suction force to catheter lumen 140 of catheter 108 to proximally withdraw at least a portion of the thrombus into distal opening 112 of catheter 108. For example, once distal portion 124 of catheter 108 is positioned proximate to a thrombus, a clinician may actuate a suction source 102 to apply a suction force to lumen 140. The suction force may be applied as distal member 134 is moved relative to catheter 108 and/or the thrombus.

In some example, the clinician may move distal member 134 while distal member 134 is in contact with the thrombus to disrupt the thrombus, e.g., macerate, cut, section, further compress, and the like, via surface disruption 216, or any of surface disruptions 216, 236, 246, 256, 266, 276, 286, and 296. In some examples, the outer cross-sectional dimension of distal member 134 may be configured to, along with the inner cross-sectional dimension of catheter lumen 140, compress the thrombus. For example, the outer cross-sectional diameter of distal member 134 may be from 80% to 95% the inner cross-section diameter of catheter lumen 140. In some examples, proximal taper 210 and retention member 232 are configured to enable the clinician may move distal member 134 rapidly distally and proximally, and with a substantial force without adversely impacting the mechanical connection between distal member 134 and elongated support member 132.

In some examples, the suction force applied to catheter lumen 140 of catheter 108 is varied over time, referring to herein as cyclical aspiration. As discussed above, during this cyclical aspiration, at least a portion of the thrombus may be pulled into contact with actuated inner member 118, thereby segmenting the thrombus into smaller pieces, which are then aspirated proximally through catheter lumen 140.

In some examples, the example technique of FIG. 9 also includes distally advancing inner member 118 through the thrombus until the distal member 134 at the distal portion of inner member 118 is positioned on a distal side of the thrombus. An aspiration fluid, such as saline or the like, may be introduced into support member lumen 138 and out proximal-facing openings 156 defined by distal member 134 to urge segmented thrombus portions toward the distal catheter opening 112 for aspiration. In some examples, control circuitry 128 is configured to determine and coordinate a rate or volume of introduced aspiration fluid with a corresponding rate or volume of aspirated patient fluid, e.g., by actuating suction source 102, fluid source 106, and/or an electronic valve 120 coupled to aspiration tubing 114 and/or irrigation tubing 116. As one non-limiting example, control circuitry 128 may be configured to actuate suction source 102 and open valve 120 such that about one volume unit of aspiration fluid is introduced via inner member 118 for every two volume units of patient fluid aspirated into discharge reservoir 104 (e.g., a 50% aspiration fluid to patient fluid ratio). The particular ratio may be variably selected and controlled by a user. As another example, control circuitry 128 is configured to approximately balance the volumes of aspiration fluid introduced and patient fluid withdrawn, e.g., such that the volumes are approximately equal.

In some examples, the example technique of FIG. 9 also includes retracting inner member 118 back into catheter lumen 140 of catheter 108 and directing another proximal fluid flow 168, as necessary, from the openings 156 on proximal-facing surface 152 of inner member 118 to flush the catheter lumen 140. The previous steps may be repeated, e.g., until the thrombus has been cleared from the patient's vasculature, before removing catheter 108 from the vasculature of the patient.

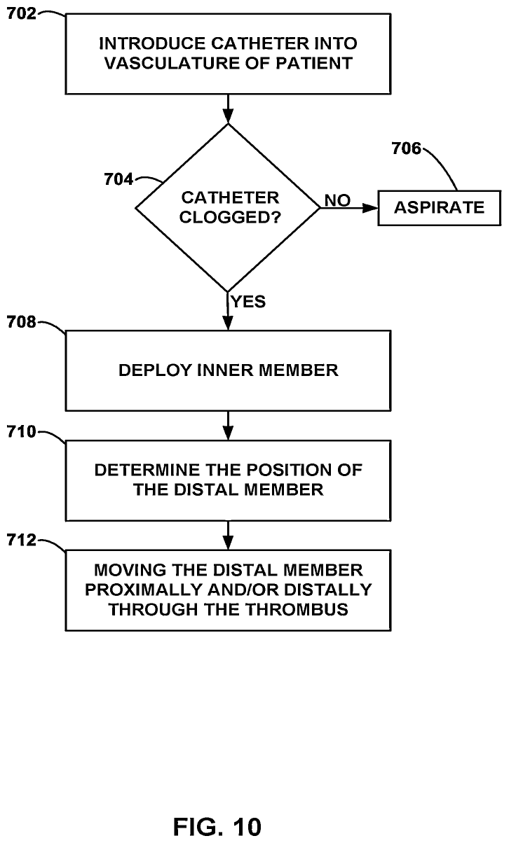

FIG. 10 is a flow diagram of another example method of using medical systems including catheters and inner members. While the example technique of FIG. 10 is described with respect to medical systems 100 and 200, catheter 108, and inner member 118 of FIGS. 1-6, the example technique of FIG. 10 may be performed using any system including an aspiration catheter and inner member described herein. The technique of FIG. 10 may be performed by any suitable user, such as a clinician, or the like.

In the example of FIG. 10, a clinician introduces aspiration catheter 108 into vasculature of a patient and distally advance aspiration catheter 108 toward a thrombus within the vasculature of the patient (702), e.g., substantially similar to (602) described above. The clinician may determine whether aspiration catheter 108 is clogged (704). For example, the flow of fluid through catheter lumen 140 may cease, the flow of fluid into a collection container configured to receive the suctioned material may slow or stop, or suction source 102 may indicate that there is clog and/or blockage of the lumen of aspiration catheter 108. If there is no clog and/or blockage of the lumen of the aspiration catheter (e.g., the fluid flows as normal), then the clinician may continue to aspirate the thrombus using aspiration catheter 108 (706).

If the clinician determines there is clog and/or blockage, the clinician deploys distal member 134 of inner member 118 from distal opening 112 of aspiration catheter 108 (708). For example, the clinician may open a hemostasis valve, e.g., of hub 126, insert inner member 118 through the hemostasis valve, and close the hemostasis valve around inner member 118 such that blood loss is reduced and/or minimized and/or reduced while still allowing for axial and/or rotational movement of inner member 118. The clinician may advance inner member 118 within catheter lumen 140 and deploy distal member 134 out of distal opening 112 from catheter lumen 140 by moving elongated support member 132. In some examples, the clinician may deploy distal member 134 to disrupt the thrombus clogging and/or blocking catheter lumen 140 and/or push and disrupt the thrombus to declog catheter lumen 140. Unclogging distal opening 112 and/or lumen 140 via inner member 118 may help expedite the aspiration procedure by at least facilitating relatively quick unblocking of the pathway for the suction force to act on the thrombus, thereby enabling efficient aspiration of the thrombus.

The clinician may determine a position of distal member 134 based on a tactile response along elongated support member 132 (710), e.g., substantially similar to (606) described above. The clinician may move the distal member 134 proximally and/or distally relative to catheter 108 to contact and disrupt the thrombus, e.g., to break the thrombus into smaller portions, to compress the thrombus or thrombus portions into smaller volumes, and/or to clear or unclog catheter lumen 140 of the thrombus, e.g., substantially similar to (608) described above.

In some examples, the clinician may deploy distal member 134 before determining whether there is a clog, e.g., prior to (704). For example, the clinician may deploy distal member 134 before or during aspiration at a target treatment site, and a thrombus may clog catheter lumen 140 with distal member 134 deployed, e.g., extended distally from catheter 108. The clinician may then declog catheter lumen 140 by first moving distal member 134 proximally to disrupt the thrombus, e.g., without pushing the thrombus from distal opening 112, and subsequently (if needed) further disrupting the thrombus by moving the distal member 134 proximally and/or distally relative to catheter 108 to contact and disrupt the thrombus, e.g., to break the thrombus into smaller portions, to compress the thrombus or thrombus portions into smaller volumes, and/or to clear or unclog catheter lumen 140 of the thrombus, e.g., substantially similar to (608) described above.

The following examples are within the scope of the present disclosure. The examples described herein may be combined in any permutation or combination.

Example 1: A medical system including: an aspiration catheter defining a catheter lumen; and an inner member configured to be received in the catheter lumen and extend distally outward from a distal opening of the catheter, wherein the inner member comprises: an elongated support member configured to move axially within the catheter lumen, the elongated support member defining a longitudinal axis; and a distal member at a distal portion of the elongated support member, wherein the distal member defines a larger cross-sectional dimension than the elongated support member in a direction orthogonal to the longitudinal axis, wherein a proximal-facing surface of the distal member defines a proximal taper, and wherein the distal member defines a surface disruption configured to aid disruption of a thrombus.

Example 2: The medical system of example 1, wherein the elongated support member defines a distal taper.

Example 3: The medical system of any of examples 1 and 2, wherein the distal member comprises a bulb including an atraumatic convex distal surface profile.

Example 4: The medical system of any of examples 1 through 3, wherein the inner member terminates at a distal end of the distal member.

Example 5: The medical system of any of examples 1 through 4, wherein the proximal taper of the distal member has an angle with reference to the longitudinal axis of 10 degrees to 30 degrees.

Example 6: The medical system of any of examples 1 through 5, wherein the proximal taper of the distal member comprises: a first taper portion having a first taper angle relative to the longitudinal axis; and a second taper portion proximal to the first taper portion, wherein the second taper portion has a second taper angle relative to the longitudinal axis, the second taper angle being less than the first taper angle.

Example 7: The medical system of any of examples 1 through 6, wherein the surface disruption comprises a notch.

Example 8: The medical system of example 7, wherein the notch comprises at least one of a continuous circumferential groove, a discontinuous circumferential groove, a longitudinal groove, a continuous spiral groove, or a discontinuous spiral groove.

Example 9: The medical system of any of examples 1 through 8, wherein the surface disruption comprises a protrusion.

Example 10: The medical system example 9, wherein the protrusion comprises at least one of a continuous circumferential ridge, a discontinuous circumferential ridge, a longitudinal ridge, a continuous spiral ridge, or a discontinuous spiral ridge.

Example 11: The medical system of any of examples 1 through 10, wherein the elongated support member comprises a retention member configured to aid attachment between the elongated support member and the distal member.

Example 12: The medical system of example 11, wherein the retention member is configured to increase a surface area of a part of the elongated support member that attaches to the distal member.

Example 13: The medical system of example 11 or example 12, wherein the retention member comprises a distal ball, wherein the distal member comprises a polymer configured to surround and attach to the distal ball.

Example 14: The medical system of any of examples 11 through 13, wherein the retention member comprises at least one of a coil, a coil anchor, a profiled wire, helix, a spiral, a spade-shape in cross-section, a slotted wire, or a T-weld.

Example 15: The medical system of any of examples 1 through 14, wherein an outer surface of the elongated support member comprises a polymer layer configured to increase an adhesion between the distal member and the outer surface of the elongated support member.

Example 16: The medical system of example 15, wherein the polymer layer comprises a polymer coating or a tubular polymer jacket.

Example 17: The medical system of any of examples 1 through 16, wherein the inner member is configured to travel a fixed distance out of the distal opening of the aspiration catheter.

Example 18: The medical system of example 17, wherein the elongated support member defines a tab configured to interact with a proximal end of the aspiration catheter to prevent further distal movement of the inner member relative to the aspiration catheter.

Example 19: The medical system of any of examples 1 through 18, wherein the inner member is biased at an angle greater than or equal to 10 degrees and less than or equal to 30 degrees relative to longitudinal axis of a proximal portion of the aspiration catheter.

Example 20: The medical system of any of examples 1 through 19, wherein the proximal taper extends to an outer surface of the elongated support member.

Example 21: The medical system of any of examples 1 through 20, wherein the proximal taper is configured to provide a tactile response along the elongated support member when the proximal taper engages with the aspiration catheter.

Example 22: The medical system of any of examples 1 through 21, wherein the proximal-facing surface of the distal member defines one or more openings configured to deliver a fluid into vasculature of a patient to dilute or displace a volume of blood aspirated from the vasculature of the patient and through the catheter lumen.

Example 23: The medical system of example 22, wherein the elongated support member comprises a tubular body defining a support member lumen configured to receive the fluid, wherein the support member lumen is fluidically coupled to the one or more openings of the distal member.

Example 24: The medical system of example 22 or example 23, wherein the elongated support member defines a support member lumen, the medical system further comprising: an aspiration pump fluidically coupled to the catheter lumen; a fluid source fluidically coupled to the support member lumen and configured to store the fluid; a valve fluidically coupled between the aspiration pump and the distal opening of the aspiration catheter; and control circuitry configured to control an opening and a closing of the valve to synchronize an aspiration force from the aspiration pump with a supply of the fluid from the fluid source.

Example 25: The medical system of example 24, further comprising an actuator switch coupled to the valve, wherein the control circuitry is configured to control the opening and the closing of the valve via the actuator switch.

Example 26: A method including: deploying a distal member of an inner member from a distal opening of an aspiration catheter and contacting a thrombus with the distal member, the aspiration catheter defining a catheter lumen, wherein the inner member comprises: an elongated support member configured to move axially within the catheter lumen, the elongated support member defining a longitudinal axis; and the distal member at a distal portion of the elongated support member, wherein the distal member defines a larger cross-sectional dimension than the elongated support member in a direction orthogonal to the longitudinal axis, wherein a proximal-facing surface of the distal member defines a proximal taper, and wherein the distal member defines a surface disruption configured to aid disruption of the thrombus; and moving, via the elongated support member, the distal member at least one of proximally or distally through the thrombus.

Example 27: The method of example 26, further comprising causing a suction source to apply a suction force to the catheter lumen to aspirate at least part of the thrombus through the catheter lumen.

Example 28: The method of example 27, wherein moving the distal member at least one of proximally or distally through the thrombus comprises moving the distal member while the suction force is being applied to the catheter lumen.

Example 29: The method of any of examples 26 through 28, further comprising: distally moving the distal member, based on determining that the position of the distal member is proximate the distal end of the catheter, to increase a suction force applied to the thrombus via the catheter lumen.

Example 30: The method of any of examples 26 through 29, wherein moving the distal member at least one of proximally or distally through the thrombus comprises repeatedly moving the distal member proximally and distally through the thrombus.

Example 31: The method of any of examples 26 through 30, wherein the elongated support member defines a distal taper.

Example 32: The method of any of examples 26 through 31, wherein the distal member comprises a bulb including an atraumatic convex distal surface profile.

Example 33: The method of any of examples 26 through 32, wherein the inner member terminates at a distal end of the distal member.

Example 34: The method of any of examples 26 through 33, wherein the proximal taper of the distal member has an angle with reference to the longitudinal axis of 10 degrees to 30 degrees.

Example 35: The method of any of examples 26 through 34, wherein the proximal taper of the distal member comprises: a first taper portion having a first taper angle relative to the longitudinal axis; and a second taper portion proximal to the first taper portion, wherein the second taper portion has a second taper angle relative to the longitudinal axis, the second taper angle being less than the first taper angle.

Example 36: The method of any of examples 26 through 35, wherein the surface disruption comprises a notch.

Example 37: The method of example 36, wherein the notch comprises at least one of a continuous circumferential groove, a discontinuous circumferential groove, a longitudinal groove, a continuous spiral groove, or a discontinuous spiral groove.

Example 38: The method of any of examples 26 through 37, wherein the surface disruption comprises a protrusion.

Example 39: The method of example 38, wherein the protrusion comprises at least one of a continuous circumferential ridge, a discontinuous circumferential ridge, a longitudinal ridge, a continuous spiral ridge, or a discontinuous spiral ridge.

Example 40: The method of any of examples 26 through 39, wherein the elongated support member comprises a retention member configured to aid attachment between the elongated support member and the distal member.

Example 41: The method of example 40, wherein the retention member is configured to increase a surface area of a part of the elongated support member that attaches to the distal member.

Example 42: The method of example 40 or example 41, wherein the retention member comprises a distal ball, wherein the distal member comprises a polymer configured to surround and attach to the distal ball.

Example 43: The method of any of examples 40 through 42, wherein the retention member comprises at least one of a coil, a coil anchor, a profiled wire, a helix, a spade-shape in cross-section, a spiral, a slotted wire, or a T-weld.

Example 44: The method of any of examples 26 through 43, wherein an outer surface of the elongated support member comprises a polymer layer configured to increase an adhesion between the distal member and the outer surface of the elongated support member.

Example 45: The method of example 44, wherein the polymer layer comprises a polymer coating or a tubular polymer jacket.

Example 46: The method of any of examples 26 through 45, wherein the inner member is configured to travel a fixed distance out of the distal opening of the aspiration catheter.

Example 47: The method of example 46, wherein the elongated support member defines a tab configured to interact with a proximal end of the aspiration catheter to prevent further distal movement of the inner member relative to the aspiration catheter.

Example 48: The method of any of examples 26 through 47, wherein the inner member is biased at an angle greater than or equal to 10 degrees and less than or equal to 30 degrees relative to longitudinal axis of a proximal portion of the aspiration catheter.

Example 49: The method of any of examples 26 through 48, wherein the proximal taper extends to an outer surface of the elongated support member.

Example 50: The method of any of examples 26 through 49, wherein the proximal taper is configured to provide a tactile response along the elongated support member when the proximal taper engages with the aspiration catheter, the method further including: determining, based on a tactile response of the elongated support member, a patency of a lumen of the aspiration catheter

Example 51: The method of any of examples 26 through 50, wherein the proximal-facing surface of the distal member defines one or more openings configured to deliver a fluid into vasculature of a patient to dilute or displace a volume of blood aspirated from the vasculature of the patient and through the catheter lumen.

Example 52: The method of example 51, wherein the elongated support member comprises a tubular body defining a support member lumen configured to receive the fluid, wherein the support member lumen is fluidically coupled to the one or more openings of the distal member.

Example 53: The method of example 51 or example 52, wherein the elongated support member defines a support member lumen, wherein an aspiration pump is fluidically coupled to the catheter lumen, wherein a fluid source is fluidically coupled to the support member lumen and configured to store the fluid, wherein a valve is fluidically coupled between the aspiration pump and the distal opening of the aspiration catheter, and wherein control circuitry is configured to control an opening and a closing of the valve to synchronize an aspiration force from the aspiration pump with a supply of the fluid from the fluid source.

Example 54: The method of example 53, further comprising an actuator switch coupled to the valve, wherein the control circuitry is configured to control the opening and the closing of the valve via the actuator switch.

Example 55: The method of example 54, further including: delivering the fluid into vasculature of the patient via the one or more openings.

Example 56: An article including: a member configured to be received in a lumen of an aspiration catheter and extend distally outward from a distal opening of the aspiration catheter, the member including: an elongated support member configured to move axially within the catheter lumen; and a distal member at a distal portion of the elongated support member, wherein the distal member defines a larger cross-sectional dimension than the elongated support member in a direction orthogonal to the longitudinal axis, wherein a proximal-facing surface of the distal member defines a proximal taper, wherein the distal member defines a surface disruption configured to aid disruption of a thrombus.

Various aspects of the disclosure have been described. These and other aspects are within the scope of the following claims.

What is claimed is:

1. A medical system comprising:
an aspiration catheter defining a catheter lumen; and
an inner member configured to be received in the catheter lumen and extend distally outward from a distal opening of the catheter, wherein the inner member comprises:
an elongated support member configured to move axially within the catheter lumen, the elongated support member defining a longitudinal axis; and
a distal member at a distal portion of the elongated support member, wherein the distal member defines a larger cross-sectional dimension than the elongated support member in a direction orthogonal to the longitudinal axis, wherein a proximal-facing surface of the distal member defines a proximal taper, and wherein the distal member defines a surface disruption configured to aid disruption of a thrombus.

2. The medical system of claim 1, wherein the elongated support member defines a distal taper.

3. The medical system of claim 1, wherein the distal member comprises a bulb including an atraumatic convex distal surface profile.

4. The medical system of claim 1, wherein the inner member terminates at a distal end of the distal member.

5. The medical system of claim 1, wherein the proximal taper of the distal member has an angle with reference to the longitudinal axis of 10 degrees to 30 degrees, wherein the proximal taper of the distal member comprises:
a first taper portion having a first taper angle relative to the longitudinal axis; and
a second taper portion proximal to the first taper portion, wherein the second taper portion has a second taper angle relative to the longitudinal axis, the second taper angle being less than the first taper angle.

6. The medical system of claim 1, wherein the surface disruption comprises at least one of a notch or a protrusion.

7. The medical system of claim 1, wherein the elongated support member comprises a retention member configured to aid attachment between the elongated support member and the distal member.

8. The medical system of claim 7, wherein the retention member is configured to increase a surface area of a part of the elongated support member that attaches to the distal member.

9. The medical system of claim 7, wherein the retention member comprises at least one of a coil, a coil anchor, a profiled wire, helix, a spiral, a spade-shape in cross-section, a slotted wire, or a T-weld.

10. The medical system of claim 7, wherein the distal member comprises a polymer configured to surround and attach to the retention member.

11. The medical system of claim 1, wherein an outer surface of the elongated support member comprises a polymer layer configured to increase an adhesion between the distal member and the outer surface of the elongated support member.

12. The medical system of claim 1, wherein the inner member is configured to travel a fixed distance out of the distal opening of the aspiration catheter.

13. The medical system of claim 1, wherein the inner member is biased at an angle greater than or equal to 10 degrees and less than or equal to 30 degrees relative to longitudinal axis of a proximal portion of the aspiration catheter.

14. The medical system of claim 1, wherein the proximal taper is configured to provide a tactile response along the elongated support member when the proximal taper engages with the aspiration catheter.

15. The medical system of claim 1, wherein the proximal-facing surface of the distal member defines one or more openings configured to deliver a fluid into vasculature of a patient to dilute or displace a volume of blood aspirated from the vasculature of the patient and through the catheter lumen.

16. The medical system of claim 15, wherein the elongated support member defines a support member lumen, the medical system further comprising:
an aspiration pump fluidically coupled to the catheter lumen;
a fluid source fluidically coupled to the support member lumen and configured to store the fluid;
a valve fluidically coupled between the aspiration pump and the distal opening of the aspiration catheter;
control circuitry configured to control an opening and a closing of the valve to synchronize an aspiration force from the aspiration pump with a supply of the fluid from the fluid source; and an actuator switch coupled to the valve, wherein the control circuitry is configured to control the opening and the closing of the valve via the actuator switch.

17. A method comprising:
deploying a distal member of an inner member from a distal opening of an aspiration catheter and contacting a thrombus with the distal member, the aspiration catheter defining a catheter lumen, wherein the inner member comprises:
an elongated support member configured to move axially within the catheter lumen, the elongated support member defining a longitudinal axis; and
the distal member at a distal portion of the elongated support member, wherein the distal member defines a larger cross-sectional dimension than the elongated support member in a direction orthogonal to the longitudinal axis, wherein a proximal-facing surface of the distal member defines a proximal taper, and wherein the distal member defines a surface disruption configured to aid disruption of the thrombus; and
moving, via the elongated support member, the distal member at least one of proximally or distally through the thrombus.

18. The method of claim 17, further comprising causing a suction source to apply a suction force to the catheter lumen to aspirate at least part of the thrombus through the catheter lumen.

19. The method of claim 17, wherein moving the distal member at least one of proximally or distally through the thrombus comprises moving the distal member while the suction force is being applied to the catheter lumen.

20. The method of claim 17, further comprising:
distally moving the distal member, based on determining that the position of the distal member is proximate the distal end of the catheter, to increase a suction force applied to the thrombus via the catheter lumen.

21. An article comprising:
a member configured to be received in a lumen of an aspiration catheter and extend distally outward from a distal opening of the aspiration catheter, the member comprising:
an elongated support member configured to move axially within the catheter lumen; and
a distal member at a distal portion of the elongated support member, wherein the distal member defines a larger cross-sectional dimension than the elongated support member in a direction orthogonal to the longitudinal axis, wherein a proximal-facing surface of the distal member defines a proximal taper, wherein the distal member defines a surface disruption configured to aid disruption of a thrombus.

22. The article of claim 21, wherein the elongated support member comprises a retention member configured to aid attachment between the elongated support member and the distal member, and wherein the retention member comprises at least one of a coil, a coil anchor, a profiled wire, a helix, a spade-shape in cross-section, a spiral, a slotted wire, or a T-weld.
